SEQUENCE LISTING (4 sequences)

SEQ ID NO: 1 - protein, 14 aa, Artificial Sequence
  Synthetic Sequence
  X (9)..(9): tryptophan or beta-(2-naphthyl)alanine
  X (14)..(14): alanine or sarcosine
IEGPTLRQXLAARX

SEQ ID NO: 2 - protein, 14 aa, Artificial Sequence
  Synthetic peptide
  X (9)..(9): beta -(2-naphthyl)alanine
  X (14)..(14): sarcosine
IEGPTLRQXLAARX

SEQ ID NO: 3 - protein, 14 aa, Artificial Sequence
  Synthetic peptide
IEGPTLRQWLAARA

SEQ ID NO: 4 - protein, 269 aa, Artificial Sequence [sequence omitted]

(19) United States
(12) Patent Application Publication
(10) Pub. No.: US 2022/0168392 A1
(43) Pub. Date: Jun. 2, 2022

(54) Methods of Enhancing Protection Against Organ and Vascular Injury, Hematopoietic Recovery and Survival in Response to Total Body Radiation/Chemical Exposure

(71) Applicant: Janssen Pharmaceutica NV, Beerse (BE)

(72) Inventors: Gary Eichenbaum, Belle Mead, NJ (US); Sanchita Ghosh, North Potomac, MD (US); Alfred Tonelli, Upper Nyack, NY (US)

(21) Appl. No.: 17/651,675

(22) Filed: Feb. 18, 2022

Related U.S. Application Data
(63) Continuation of application No. 16/752,559, filed on Jan. 24, 2020.
(60) Provisional application No. 62/796,728, filed on Jan. 25, 2019.

(51) Int. Cl. A61K 38/19; A61K 47/60; A61P 39/00; A61K 45/06

(52) U.S. Cl. CPC A61K 38/196; A61K 45/06; A61P 39/00; A61K 47/60

(57) ABSTRACT

Methods of mitigating vascular injury, promoting organ and hematopoietic recovery, accelerating vascular recovery, and enhancing survival in a subject treated with radiation therapy or chemotherapy are described. In particular, an effective amount of a thrombopoietin (TPO) mimetic, such as RWJ-800088, is used at the appropriate times relative to the Total Body Irradiation or Chemotherapy exposure to achieve these prophylactic and/or therapeutic benefits.

### CROSS REFERENCE TO RELATED APPLICATIONS

This application claims the benefit to U.S. patent application Ser. No. 16/752,559 filed Jan. 24, 2020, which claims benefit to U.S. Provisional Patent Application No. 62/796,728, filed Jan. 25, 2019, the disclosures of which are hereby incorporated by reference herein in its entirety.

### STATEMENT OF GOVERNMENT INTEREST

This invention was made with government support under the National Institute of Allergy and Infectious Diseases AA112044-001-04000 Grant; Defense Medical Research and Development Program JPC-7 project DM178020, and the Armed Forces Radiobiological Research Institute Intramural funding RAB23338. The Government has certain rights in the invention.

### REFERENCE TO SEQUENCE LISTING SUBMITTED ELECTRONICALLY

This application contains a sequence listing that is submitted electronically via EFS-Web as an ASCII formatted sequence listing with a file name “004852-102US3 Sequence Listing,” creation date of Feb. 18, 2022, and having a size of about 4 KB. The sequence listing submitted via EFS-Web is part of the specification and is herein incorporated by reference in its entirety.

### FIELD OF THE INVENTION

This invention relates to methods of mitigating vascular injury, promoting organ recovery, and/or enhancing survival in a subject exposed to or medically treated with whole body radiation or systemic radio or chemotherapy. The methods comprise administering to the subject an effective amount of a thrombopoietin (TPO) mimetic before (preferred), during or following the radiation or chemical exposure to achieve these prophylactic and/or therapeutic benefits.

### BACKGROUND OF THE INVENTION

Acute radiation syndrome (ARS), also known as radiation toxicity or radiation sickness, is an acute illness caused by irradiation of the entire body (or most of the body) by a high dose of penetrating radiation in a very short period of time. It is a multi-phasic process that can lead to morbidity and mortality (Waselenko et al., Ann. Intern. Med. 140(12):1037-51 (2004)). Immediate effects of irradiation are seen within the vasculature followed by pronounced hematopoietic effects (Krigsfeld et al., Radiat. Res. 180(3): 231-4 (2013)). Within 24 hours after irradiation, vascular endothelial cells express adhesion molecules (e.g. L-selectin), which promotes leukocyte adhesion and extravasation, and can lead to an inflammatory response (Hallahan et al., Biochem. Biophys. Res. Commun. 217(3):784-95 (1995); Hallahan et al., Radiat. Res. 152(1):6-13 (1999)). These early vascular effects can be accompanied by exposure of the basement membrane and hemorrhage, which then leads to microclot formation. Depending on the extent of microclot formation, this can then lead to platelet and fibrinolytic factor depletion. Because the platelet population cannot be efficiently replenished due to irradiation depletion of hematopoietic stem cells, thrombocytopenia ensues, leading to further vascular thinning and a progression to disseminated intravascular coagulopathy (DIC). Therefore, protection of the vascular endothelium can mitigate radiation-induced injury and mortality (Rotolo et al., J. Clin. Invest. 122(5):1786-90 (2012)). Similar toxicities and mortality are observed in patients that are undergoing myeloablative bone marrow transplant conditioning with total body irradiation (TBI) and chemotherapy (Kebriaei, P, et al. Blood, Volume 128(22):679-679, Dec. 2, 2016).

The pathogenesis leading to injury to surrounding normal tissue following radiation exposure is complex. Ionizing radiation causes cell death, both parenchymal and vascular, through direct cytotoxicity (excessive generation of reactive oxygen species), inflammation, and the innate immune response (Kim et al., Radiat. Oncol. J. 32(3):103-15 (2014)). Some changes occur acutely, (i.e., inflammation, vascular endothelial injury, micro-hemorrhage) while others manifest weeks to months after radiation exposure (i.e., chronic inflammation, nerve dysfunction, scarring and fibrosis). Fibroblast proliferation is a key component of later stage radiation therapy (RT) injury (Brush et al., Semin. Radiat. Oncol. 17(2):121-30 (2007)).

Given that multiple facets of radiation-induced morbidity and mortality stem from platelet depletion, several investigators have evaluated whether thrombopoietin (TPO)-based therapies are effective in preventing acute radiation syndrome (Mouthon et al., Can. J. Physiol. Pharmacol. 80(7):717-21 (2002); Neelis et al., Blood 90(7):2565-73 (1997)). Potential mechanisms for the enhanced survival include its myeloprotective and platelet stimulatory effects as well as direct protective and/or reparative effects on vascular endothelium. Thrombopoietin (TPO) is a growth factor that is synthesized and secreted by the liver. TPO regulates platelet levels by binding to c-mpl on megakaryocytes (to stimulate platelet maturation) and existing platelets (providing negative feedback) (Mitchell and Bussell, Semin. Hematol. 52(1):46-52 (2015)). TPO can also act directly on vascular endothelial cells and cardiomyocytes by binding to c-mpl receptors located on these cells (Langer et al., J. Mol. Cell Cardiol. 47(2):315-25 (2009)). There have been several studies demonstrating direct vascular protective effects of thrombopoietin in animal models of doxorubicin mediated cardiovascular injury (Chan et al., Eur. J. Heart Fail. 13(4):366-76 (2011)), cardiovascular ischemia reperfusion injury (Baker et al., Cardiovasc. Res. 77(1):44-53 (2008)) and stroke (Zhou et al., J. Cereb Blood Flow Metab. 31(3):924-33 (2011)). Recombinant human TPO is not a viable therapy in humans, however, due to induction of cross-reactive antibodies to endogenous TPO that can lead to chronic thrombocytopenia (Li et al., Blood 98(12):3241-8 (2001)).

Total body irradiation (TBI) is a powerful but potentially hazardous tool used before bone marrow transplantation (BMT) (Gyurkocza, B., et al., Blood. 2014; 124(3): 344-353) in the treatment of malignant disorders and some non-malignant hematological and metabolic conditions. Side effects of TBI include short-term side effects, such as headache, nausea and vomiting, diarrhea, fatigue, skin reactions, infection and bone marrow suppression (e.g., low blood count), as well as mid to long-term side effects, such as Graft versus Host Disease (Newell, L, et al., Blood 2016 128 22 63), Veno Occlusive Disease (Deode, T, et al., Biol. Blood Marrow Transplant 23 (2017) S18-S39, Thrombotic Microangiopathy (Khosla, J Bone Marrow Transplantation (2017) 00, 1-9), growth and endocrine dysfunction, organ specific damage, second malignancy (see, e.g., Leiper, Arch Dis Child. 1995 Can; 72(5): 382-385). Other sequelae from chemotherapy and therapeutic application of TBI which are associated with vascular damage includes but are not limited to: mucositis, associated with defined therapies for neoplasms of the head and neck; chemotherapy-related cognitive impairment (i.e., chemo brain fog), which develops in a percentage of women receiving therapies for metastatic breast cancer; focal vascular lesions including petechiae, cellulitis, ecchymosis and generalized tissue hemorrhage including gastrointestinal hemorrhaging and chemotherapy induced lesions of the tongue.

Chemotherapies, such as radiomimetic chemotherapies, are used for the treatment of malignant disorders and during bone marrow transplant conditioning regimens (Gyurkocza, B., et al., Blood. 2014; 124(3): 344-353). They, too, have similar side effects. The concurrent administration of chemotherapy and radiotherapy can result in side effects worse than each therapy alone.

There exists an important and unmet need for methods and compositions that provide enhanced protection of normal tissues without decreasing the efficacy of radiation therapy and/or chemotherapy and enhanced survival following exposure to lethal doses of radiation.

### BRIEF SUMMARY OF THE INVENTION

In one general aspect, provided herein is a method of mitigating vascular injury, protecting against organ and hematopoietic injury, promoting functional organ and hematopoietic recovery, accelerating vascular recovery, and enhancing survival in a human subject exposed to whole body radiation and/or chemotherapy. The method comprises subcutaneously or intravenously administering to the human subject of an effective amount of a thrombopoietin (TPO) mimetic comprising the amino acid sequence of SEQ ID NO:1, wherein the effective amount comprises 0.1 microgram (μg) to 6 μg, preferably 2.25 μg to 4 μg, of the TPO mimetic per kilograms (kg) body weight of the subject, or the fixed or tiered dose equivalents based upon a typical body weight of the subject population. In another general aspect, provided is a method of treating a human subject in need of eradication of malignant cells and/or conditioning the bone marrow to enable bone marrow transplantation. The method comprises: a) treating the human subject with at least one of a radiation therapy and a radiomimetic chemotherapy, and b) subcutaneously or intravenously administering to the human subject an effective amount of a thrombopoietin (TPO) mimetic comprising the amino acid sequence of SEQ ID NO:1, wherein the effective amount comprises 0.1 microgram (μg) to 6 μg, preferably 2.25 μg to 4 μg, of the TPO mimetic per kilograms (kg) body weight, or the fixed or tiered dose equivalents based upon a typical body weight of the subject population. According to embodiments of the application, the TPO mimetic is administered within a time period of about 32 hours before to about 24 hours after the subject is exposed to the at least one of radiation and chemotherapy, preferably the TPO mimetic is administered about 32 hours to 10 minutes before the subject is exposed to the radiation and/or chemotherapy.

Another general aspect of the invention relates to a method of mitigating vascular and hematopoietic injury, promoting organ and/or hematopoietic recovery, enhancing survival, and/or protecting against organ and hematopoietic injury in a subject exposed to either radiation radiomimetic chemotherapy or both. The method comprises administering to the subject an effective amount of a thrombopoietin (TPO) mimetic comprising the amino acid sequence of SEQ ID NO:1, wherein the TPO mimetic is administered to the subject within about 32 hours prior to the subject being treated with either radiation therapy, radiomimetic chemotherapy or both. Yet another general aspect of the invention relates to a method of treating a subject in need of eradication of malignant cells. The method comprises: a) treating the human subject with at least radiation therapy or radiomimetic chemotherapy, or both and b) administering to the subject an effective amount of a thrombopoietin (TPO) mimetic comprising the amino acid sequence of SEQ ID NO:1, wherein the TPO mimetic is administered to the subject within about 32 hours prior to the subject being treated with the at least one of the radiation therapy and the radiomimetic chemotherapy. According to embodiments of the application, the effective amount of TPO mimetic is administered to the subject subcutaneously, and when the subject is a human being, the effective amount of the TPO mimetic is about 0.1 microgram (μg) to about 6 μg, preferably 2.25 μg to 4 μg, of the TPO mimetic per kilograms (kg) body weight of the subject, or the fixed or tiered dose equivalents based upon a typical body weight of the subject population; when the subject is a mouse, the effective amount of the TPO mimetic is about 100 μg to about 5,000 μg/kg body weight of the subject; when the subject is a rat, the effective amount of the TPO mimetic is about 1,000 μg to about 5,000 μg/kg body weight of the subject; or when the is a dog or a monkey, the effective amount of the TPO mimetic is about 10,000 μg to about 50,000 μg/kg body weight of the subject. The reason for these differences in dose across species to achieve comparable therapeutic benefit is due to species differences in potency of TPOm. The TPOm dose required to achieve maximal platelet elevation in humans, mice, rats, canines and rhesus macaques is shown in Table 1. The dose required to achieve a 2-3-fold elevation in humans is ˜100-fold lower compared to mice, ˜1,000-fold lower compared to rats and >10,000-fold lower compared to canines and NHPs. The maximum platelet elevation was greater than 3-fold for all species except canine, in which the maximum platelet elevation was ˜1.7 fold, suggesting that the canine is the least responsive species.

TABLE 1
Doses that produce the maximum
platelet elevation relative to baseline in
normal healthy animals and humans.

| | | Average of the |
|---|---|---|
| | Dose | Maximum Platelet Elevation/ |
| Species | (μg/kg) | Baseline Platelet Counts |
| Human | 3 | 3.3 |
| Mouse | 300 | 3.9 |
| Rat | 3000 | 3.1 |
| Dog | 10000 | 1.7 |
| Monkey | 40000 | 3.83 |

The species differences in potency has been described for other TPO mimetics in the literature and is attributed to differences in receptor affinity (Erickson-Miller C L, et al. “Discovery and characterization of a selective, non-peptidyl thrombopoietin receptor agonist,” Exp. Hematol., 2005; 33:85-93 and Nakamura T, et al. “A novel non-peptidyl human c-Mpl activator stimulates human megakaryopoiesis and thrombopoiesis,” Blood. 2006; 107:4300-7). Despite the differences in dose across species, there is clear evidence that there is a comparable maximum platelet response across the human, mouse, rat and monkey with a TPO mimetic comprising the amino acid sequence of SEQ ID NO:1.

In certain embodiments of the application, the TPO mimetic is RWJ-800088 having the following structure of formula (I), or a pharmaceutically acceptable salt or ester thereof:

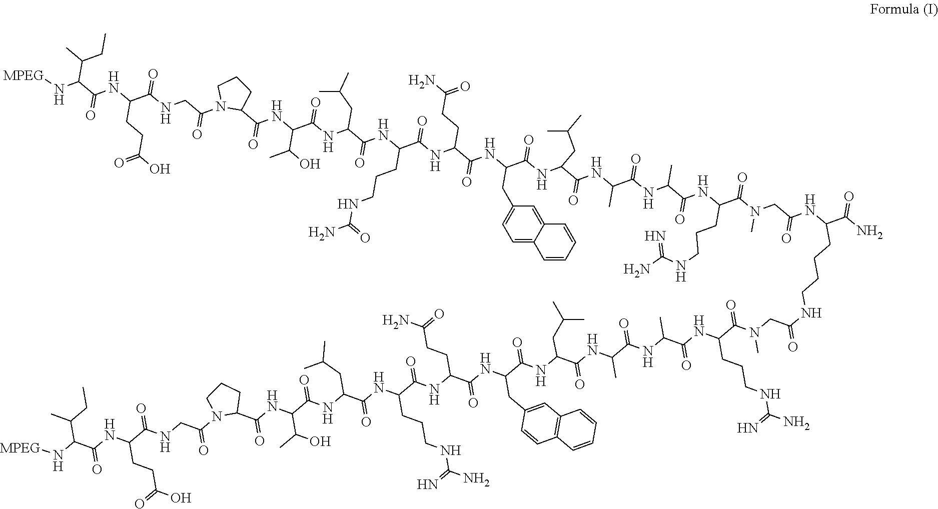

wherein MPEG represents methoxypolyethyleneglycol20000.

In certain embodiments of the application, the TPO mimetic is romiplostim comprising the amino acid sequence of SEQ ID NO:4.

In certain embodiments of the application, the subject is treated for mitigation of toxicity and enhancement of survival for Acute Radiation Syndrome or radiation and/or chemotherapy used in bone marrow transplant conditioning.

In certain embodiments of the application, the subject is treated for a cancer selected from the group consisting of a leukemia, multiple myeloma, acute lymphocytic leukemia, a solid tumor, Morbus Hodgkin's disease and Non-Hodgkin's lymphomas. The subject is treated with total body irradiation prior to transplantation of at least one of haematopoietic stem cells, bone marrow stem cells and peripheral blood progenitor stem cells.

In certain embodiments of the application, the subject is treated with a radiomimetic chemotherapy selected from the group consisting of ozone, peroxide, an alkylating agent, an antimetabolite agent, a platinum-based agent, a cytotoxic antibiotic, and a vesicant chemotherapy, preferably, the radiomimetic chemotherapy is alkylating agents (busulfan, melphalan, carmustine, cyclophosphamide, thiotepa), antimetabolite class of agents (fludarabine, clofrabine, cytarabine, 6-thioguanine), topoisomerase II inhibitor (etoposide), and/or a platinum-based agent selected from the group consisting of cisplatin, carboplatin, oxaliplatin, and nedaplatin. The chemotherapy is administered to the subject alone or in combination with a targeted radiation therapy or total body irradiation.

In certain embodiments of the application, the subject is administered a single dose of the effective amount of the TPO mimetic.

In certain embodiments of the application, the subject is administered more than one dose of the effective amount of the TPO mimetic.

Another general aspect of the application relates to a method of treating a cancer in a human subject in need of, comprising: (a) treating the human subject with at least one of a radiation therapy and a radiomimetic chemotherapy, and (b) subcutaneously administering to the human subject an effective amount of a thrombopoietin (TPO) mimetic comprising RWJ-800088 or romiplostim, wherein the effective amount comprises 0.5 microgram (μg) to 5 μg, preferably 2.25 μg to 4 μg, of the TPO mimetic per kilograms (kg) body weight of the subject, or the fixed or tiered dose equivalents based upon a typical body weight of the subject population, and the TPO mimetic is administered to the subject within a time period from about 32 hours prior to the subject being treated with the at least one of the radiation therapy and/or the radiomimetic chemotherapy to immediately after the treatment. In other embodiments, the subject is treated for a cancer selected from the group consisting of a leukemia, a solid tumor, Morbus Hodgkin's disease and Non-Hodgkin's lymphomas, and the subject is treated with total body irradiation prior to transplantation of at least one of haematopoietic stem cells, bone marrow stem cells and peripheral blood progenitor stem cells. In further embodiments, the subject is treated with a radiomimetic chemotherapy selected from the group consisting of ozone, peroxide, an alkylating agent, a platinum-based agent, a cytotoxic antibiotic, and a vesicant chemotherapy, preferably, the radiomimetic chemotherapy is ozone, peroxide, an alkylating agent, an antimetabolite agent, a platinum-based agent, a cytotoxic antibiotic, and a vesicant chemotherapy, preferably, the radiomimetic chemotherapy is alkylating agents (busulfan, melphalan, carmustine, cyclophosphamide, thiotepa), antimetabolite class of agents (fludarabine, clofrabine, cytarabine, 6-thioguanine), topoisomerase II inhibitor (etoposide), and/or a platinum-based agent selected from the group consisting of cisplatin, carboplatin, oxaliplatin, and nedaplatin. In some embodiments, the subject is administered a single dose of the effective amount of the TPO mimetic. In other embodiments, the subject is administered more than one dose of the effective amount of the TPO mimetic. In certain embodiments, the effective amount of the TPO mimetic is about 0.5 μg/kg, 1 μg/kg, 1.25 μg/kg, 1.5 μg/kg, 1.75 μg/kg, 2 μg/kg, 2.25 μg/kg, 2.5 μg/kg, 2.75 μg/kg, 3 μg/kg, 3.25 μg/kg, 3.5 μg/kg, 3.75 μg/kg, 4 μg/kg, 4.25 μg/kg, 4.5 μg/kg, 4.75 μg/kg, 5 μg/kg, or any amount in between, of body weight of the subject, or the fixed or tiered dose equivalents based upon a typical body weight of the subject population. In preferred embodiments, the effective amount of the TPO mimetic is about 2 μg/kg, 2.25 μg/kg, 2.5 μg/kg, 2.75 μg/kg, 3 μg/kg, 3.25 μg/kg, 3.5 μg/kg, 3.75 μg/kg, 4 μg/kg of body weight of the subject, or the fixed or tiered dose equivalents based upon a typical body weight of the subject population. In certain embodiments, the effective amount of the TPO mimetic is administered to the subject about 32, 28, 24, 20, 16, 12, 8, 4, 3, 2, 1, 0.5, 0.1 hours, or anytime in between, prior to the subject being treated with the at least one of the radiation therapy and the radiomimetic chemotherapy.

### BRIEF DESCRIPTION OF THE DRAWINGS

The patent or application file contains at least one drawing executed in color. Copies of this patent or patent application publication with color drawings will be provided by the Office upon request and payment of the necessary fess.

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A shows the protective effect of RWJ-800088 administration on carboplatin induced thrombocytopenia (platelets) in the mouse, and FIG. 1B shows the protective effect (reduced decrease in RBCs) of RWJ-800088 administration on carboplatin induced anemia (RBC) in the mouse.

FIG. 2 shows the effect of RWJ 800088 on the development of carboplatin induced microangiopathic events in the brains of mice.

FIGS. 3A-D show survival following single SC doses of RWJ-800088 administered 24 h prior to TBI in CD2F1 male mice (n=24/group) and supralethal doses of TBI (0.6 Gy/min) at 9.35 Gy (FIG. 3A), 9.75 Gy (FIG. 3B), 10.5 Gy (FIG. 3C) and 11.0 Gy (FIG. 3D).

FIGS. 4A-G shows the enhanced recovery of peripheral blood cells (white blood cells (WBCs), red blood cells (RBCs), % hematocrit (% HCT), neutrophils, platelets (PLT), monocytes (MON) and lymphocytes (LYM)) when 0.3 mg/kg of RWJ-800088 was administered 24 h prior to TBI. Day 0 represents day of irradiation. Blood was collected on days 0 (2 hours after exposure), 1, 3, 7, 10, 14, 21, and 30 post-TBI (7 Gy). Data represented are mean±standard error of the mean (SEM) for n=10 mice. Significant difference (p<0.001-0.0125) between RWJ-800088 treated and saline treated irradiated groups by ANOVA is indicated with an asterisk (*). The inlayed sub-plots refer to the Day 10 and 14 counts in the treated vs. vehicle. Some data points in the figure do not have error bars that are visible because they are smaller than the symbols.

FIGS. 5A-B show the circulating levels of erythropoietin (FIG. 5A) and FLT-3 ligand (FIG. 5B) in mice pre-treated with RWJ-800088 vs. saline at 24-hours pre-TBI. Circulating levels in control mice that did not receive TBI or RWJ-800088 are shown on Day 0. *** indicates p-value <0.0001.

FIGS. 6A-D show the circulating levels of MMP9 (FIG. 6A), VCAM1 (FIG. 6B), E-Selectin (FIG. 6C) and sP-Selectin (FIG. 6D) in mice pre-treated with RWJ-800088 vs. saline at 24-hours pre-TBI. Circulating levels in control mice that did not receive TBI or RWJ-800088 are shown on Day 0. *** indicates p-value <0.0001.

FIG. 7 shows the enhanced recovery of hematopoietic progenitor cells after a non-lethal dose of radiation (7 Gy) in CD2F1 mice (n=6 per group) when RWJ-800088 was administered 24 h prior to TBI. Clonogenic potential of bone marrow cells was assessed by a CFU assay. Colony forming units (CFU) were assayed on days 0 (2 h post-TBI), 1, 3, 7, 15, and 30 after exposure. Cells from three femurs were pooled, counted, and each sample plated in duplicate to be scored 14 days after plating. Data are expressed as mean±Standard error of mean (SEM). Statistical significance was determined between irradiated saline treated and RWJ-800088 treated groups.

FIG. 8 shows the sternal bone marrow hematopoietic cell recovery after non-lethal dose of TBI (7 Gy) when administered 24 h prior to TBI. Representative sternal bone marrow sections are shown for non-irradiated vehicle (NRV) and RWJ-800088 (NRD) treated mice from days 0 and 30, and from saline (RV) and RWJ-800088 (RD) treated irradiated mice from days 0 (2 h post-TBI), 1, 3, 7, 15 and 30 post-TBI. Bone marrow cellularity and megakaryocyte numbers were quantitated from histological sections from days 0, 1, 3, 7, 15 and 30. Significant increase in bone marrow cellularity and megakaryocytes were observed after 7 days post-TBI in the RWJ-800088 treatment group. Even after 15 days post-TBI, there was significant difference in cellularity of saline treated and RWJ-800088 treated groups, later showing recovery from radiation injury. Data represented are mean t standard error of the mean (SEM) for n=6 mice. Percent (%) range of Cellularity: Grade 1: <10%; Grade 2: 11-30%; Grade 3: 31-60%; Grade 4: 61-89%4; Grade 5: >90%.

FIG. 9A shows a 100% improvement in survival in the animals treated with RWJ-800088 (1 mg/kg) compared to Vehicle (saline), when they were administered 24 h prior to a supra-lethal dose of TBI (11 Gy) in CD2F1 male mice (8 mice/group/time point). FIG. 9B shows visually that recovery from gastrointestinal injury is accelerated with RWJ-800088 compared to Vehicle. FIG. 9C shows that the number of viable crypts is increased with RWJ-800088 compared to Vehicle. Data are expressed as mean±Standard error of mean (SEM). Irradiated TPOm group compared with saline treated group, statistical significance by Student T test (p≤0.0001)

FIGS. 10A and 10B show a significant reduction of bacterial translocation to liver (FIG. 10A) and spleen (FIG. 10B) when RWJ-800088 (1 mg/kg) was administered 24 h pre-TBI (12.5 Gy) to male CD2F1 mice (8/group/time point). Bacterial translocation was determined as bacterial load in liver and spleen tissue as a result of radiation injury to the intestines and was quantified by real-time polymerase chain reaction (PCR) using the 16S rRNA gene consensus sequence. Naïve jejunum serves as a positive control and naïve spleen and liver serve as negative controls/baseline values. Data are expressed as mean±Standard error of mean (SEM). Irradiated TPOm group compared with saline treated group, statistical significance by Student T test (p≤0.0001).

FIGS. 11A and 11B show that biomarkers for sepsis (Serum Amyloid A (SAA)—FIG. 11A and Procalcitonin (PCT)—FIG. 11B) were reduced on Day 9 when RWJ-800088 (1 mg/kg) was administered 24 hours prior to TBI (11 Gy) of CD2F1 male mice (8 mice/group/time point). Data are expressed as mean±Standard error of mean (SEM). Irradiated TPOm group compared with saline treated group, statistical significance by Student T test (p≤0.0001). Serum amaloyd A (SAA) and pro-calcitonin (PCT), sepsis markers, were both measured by ELISA.

FIG. 12A shows the dose response for survival following single SC doses of RWJ-800088 (0.1-3.0 mg/kg) or Vehicle (Saline) administered 24 hours post-TBI (9.3-9.35 Gy) to CD2F1 male mice from several studies (n=24/dose except for at 0.3 mg/kg where n=120 and 1 mg/kg where n=48) and FIG. 12B shows the comparison of single vs. multiple dose regimens of RWJ-800088 (0.3 kg/kg) administered SC at 24 hours post TBI (9.35 Gy) on survival vs. time in lethally irradiated CD2F1 male mice (n=24/group).

FIG. 13 shows the impact of timing of RWJ-80088 (1 mg/kg) administration relative to TBI (9.35 Gy for 24 hour post and 9.75 Gy for all other time points) on survival of CD2F1 mice (n=24/group except for the 4 hour time point where n=48).

FIG. 14 shows the dose reduction factor for RWJ-800088 administered 24 hours post-TBI to CD2F1 male mice (n=24/time point)

FIG. 15 shows the dose reduction factor for RWJ-800088 administered 24 hours pre-TBI to CD2F1 male mice (n=24/time point)

FIGS. 16A-B show the percent survival in female SD Rats (n=8/group) administered RWJ-800088 (TPOm) at different time points and dose levels—FIG. 16A: 3000 μg/kg at 6, 24 and 48 hours post TBI (7.18 Gy), and FIG. 16B: 300 and 3000 μg/kg at 24 hours post TBI (7.18 Gy).

FIG. 17 shows the survival curve from a pilot Study in Rhesus monkeys (n=10/group−5 male/5 female) treated with vehicle or RWJ-800088 at a 30 mg/kg single dose at 24 hours post irradiation following TBI gamma radiation (600 cGY).

FIGS. 18A-D shows plots of the platelet (FIG. 18A), red blood cell (FIG. 18B), reticulocytes (FIG. 18C), and white blood cell (FIG. 18D) following administration of RWJ-800088 (RWJ-800088) (30 mg/kg single dose at 24 hours post-TBI (600 cGY) to Rhesus monkeys (n=5 male and 5 female).

FIG. 19 shows the platelet count (10⁹/L) following placebo or a single IV dose of RWJ-800088 (ranging between 0.375 to 3 μg/kg) in healthy male volunteers (n=6/group for treatment and 10/placebo).

FIG. 20 shows the mean change of platelet counts from baseline=0 (mean±1 SE) following placebo or a single IV dose of RWJ-800088 (1.5 μg/kg, 2.25 μg/kg, and 3 μg/kg) given at least 2 hours (but not more than 4 hours) prior to each of 2 cycles of platinum-based chemotherapies in cancer patients receiving 2-cycles of platinum-based chemotherapy 21 days apart (Study NAP1002).

FIG. 21 shows the mean change of hemoglobin counts from baseline (mean±1 SEM) following placebo or a single IV dose of RWJ-800088 (1.5 μg/kg, 2.25 μg/kg, and 3 μg/kg) in cancer patients receiving platinum-based chemotherapy receiving 2-cycles of platinum-based chemotherapy 21 days apart (Study NAP1002)

FIGS. 22A-E show the increase of different types of blood cells in mice administered with RWJ-800088 compared to those administered with vehicle at 6 months and 12 months post TBI including white blood cells (FIG. 22A), lymphocytes (FIG. 22B), neutrophils (FIG. 22C), platelets (FIG. 22D), and red blood cells (FIG. 22E) in Experiment 5.

FIG. 23 shows the increase of colony forming units in isolated bone marrow in mice administered with RWJ-800088 compared to those administered with vehicle at 6 months post TBI in Experiment 5.

FIG. 24 shows the increase of megakaryocytes in mice administered with RWJ-800088 compared to those administered with vehicle at 1 month and 6 months post TBI in Experiment 5.

FIG. 25 shows immunofluorescence evaluating kidney slides stained with β-catenin or E-cadherin at 1 month and 6 months post TBI in Experiment 5.

FIG. 26 shows immunofluorescence evaluating kidney slides stained with β-galactosidase at 1 month and 6 months post TBI in Experiment 5.

FIG. 27 shows the increase of cells stained positive for β-galactosidase in mice administered with RWJ-800088 compared to those administered with vehicle at 1 month and 6 months post TBI in Experiment 5.

### DETAILED DESCRIPTION OF THE INVENTION

This disclosure is based upon, at least in part, on the identification of a thrombopoietin (TPO) mimetic as a therapeutic for protecting against vascular injury, promoting organ and hematopoietic recovery, and/or enhancing vascular recovery and survival in subjects exposed to a targeted or whole body lethal and supra-lethal doses of radiation or chemotherapy. The TPO mimetic can be formulated and administered to subjects exposed to radiation or chemotherapy to protect against the negative effects of the radiation or chemotherapy on the vasculature or bone marrow and to increase the overall chances for survival of the subject.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains,” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human, to who will be or has been vaccinated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right”, “left”, “lower” and “upper” designate directions in the drawings to which reference is made.

As used herein, the term “in combination”, in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “mitigating vascular injury,” as used herein, refers to improving and restoring at least one of the normal functions and structures of the vascular system in a subject following radiation or radiochemotherapy. A primary function of the vascular system is to carry blood and lymph throughout the body of the subject, delivering oxygen and nutrients and taking away tissue waste matter. The term “mitigating vascular injury” can refer to improving or restoring the affected function of the vascular system such that the circulation of blood and lymph throughout the body is not altered significantly upon exposure to the radiation therapy (RT) or chemotherapy. The term “mitigating vascular injury” can also refer to preserving or maintaining one or more other functions of the vascular system, such as protecting the subject from impaired pudendal artery vasodilation following RT or chemotherapy, or reducing vasoconstriction following RT or chemotherapy. The vascular system is composed of blood vessels (e.g., arteries, veins, and capillaries) and lymph vessels that circulate the blood and lymph, respectively, throughout the body. Structurally, blood vessels are composed of an outer endothelium layer and three tissue layers; the tunica externa, the tunica media, and the tunica intima. The term “mitigating vascular injury” can further refer to preserving or maintaining the structure of the vascular system such that the structure of the vascular system is not altered or affected significantly following RT or chemotherapy, for example, there is no substantial vascular leakage or substantial increase in vascular endothelial leukocyte interaction following the RT or chemotherapy.

The term “hematopoietic injury,” as used herein, refers to the injury to the hematopoietic system in a subject following RT or chemotherapy, which is primarily due to the onset of apoptosis in bone marrow cells and bone marrow hematopoietic stem cells. Hematopoietic injury includes, but is not limited to, lymphocytopenia, neutropenia, thrombocytopenia, anemia, and possible death from infection and/or hemorrhage.

The term “hematopoietic recovery,” as used herein, refers to the restoration of normal functions and structures of the hematopoietic system in a subject following radiation or radiochemotherapy. It also includes the recovery after bone barrow transplant. Hematopoietic recovery can be determined using methods known in the art in view of the present disclosure. Examples of the methods are, but are not limited to, platelet counts, red blood cell (RBL) counts, reticulocyte counts, hemoglobin concentration [HGB], hematocrit concentration [HCT], as well as immunohistochemical analysis of microangiopathic events.

The term “vascular recovery,” as used herein, refers to the restoration of normal functions and structures of the vascular system in a subject following radiation or radiochemotherapy. Vascular recovery can be determined using methods known in the art in view of the present disclosure.

The term “organ injury,” as used herein, refers to the injury to one or more organs in a subject following RT or chemotherapy, which is primarily due to the reduction of production of blood cells and/or damage to the digestive tract. Organ injury includes, but is not limited to, the damages to heart and blood vessels (cardiovascular system), brain, skin, gastrointestinal tract, liver, spleen, or bone marrow. Examples of the organ injury are hemorrhage or edema in the brain, intestinal discomfort, stomach sores, bacterial translocation to liver or spleen, infertility, cardiovascular disease, and hypopituitarism.

The term “organ recovery,” as used herein, refers to the restoration of normal functions and structures of the affected organs in a subject following radiation or radiochemotherapy.

Radiation Therapy

The term “radiation therapy” or “RT”, as used herein, refers to a therapy using ionizing radiation to control cell growth. It is generally used as part of cancer treatment. Radiation therapy (RT) is sometimes also referred to as radiation treatment, radiotherapy, irradiation, or x-ray therapy. Radiation therapy includes, but is not limited to, targeted radiation, and total body irradiation therapy.

Targeted Radiation Therapy

The term “TRT” or “targeted radiation therapy”, as used herein, refers to a therapy using ionizing radiation, or a radiomimetic agent, that is preferentially targeted or localized to a specific organ or part of the body. It is generally used as part of cancer treatment. Targeted radiation therapy (TRT), such as targeted ionizing radiation therapy, is sometimes also referred to as radiation treatment, radiotherapy, irradiation, or x-ray therapy. There are three main divisions of targeted radiation therapy: external beam radiation therapy (EBRT or XRT), internal radiation therapy, and systemic radioisotope therapy. Sometime, the radiation can be given in several treatments to deliver the same or slightly higher dose, which is called fractioned radiation therapy.

External beam radiation therapy (EBRT) uses a machine that directs high-energy rays from outside the body into the tumor. Examples of EBRT include, but are not limited to, stereotactic radiation therapy, image guided radiation therapy (IGRT), intensity modulated radiation therapy (IMRT), helical-tomotherapy, proton beam radiation therapy, and intraoperative radiation therapy (IORT).

Internal radiation is also called brachytherapy, in which a radioactive implant is put inside the body in or near the tumor. It allows a higher dose of radiation in a smaller area than might be possible with external radiation treatment. It uses a radiation source that's usually sealed in a small holder called an implant. Different types of implants can be called pellets, seeds, ribbons, wires, needles, capsules, balloons, or tubes. One such example of internal radiation is transarterial chemoembolization (TACE).

Systemic radioisotope therapy (SRT) is also called unsealed source radiotherapy. Targeted radioactive drugs are used in SRT to treat certain types of cancer systemically, such as thyroid, bone, and prostate. These drugs, which are typically linked to a targeting entity—such as a monoclonal antibody or a cell-specific ligand, can be given by mouth or put into a vein; they then travel through the body until reaching at the desired target, where the drug will accumulate in a relatively high concentration.

Total Body Irradiation

Total body irradiation (TBI), also referred as whole-body radiation therapy, is another form of radiation therapy, which involves irradiation of the entire body. TBI is used primarily as part of the preparative regimen for transplantation of hematopoietic stem cell, bone marrow stem cells or peripheral blood progenitor stem cells, in the treatment of hematopoietic diseases. TBI is done to kill any cancer cells that are left in the body and helps make room in the patient's bone marrow for new blood stem cells to grow. TBI also helps prevent the body's immune system from rejecting the transplanted stem cells.

The indications for TBI include, but are not limited to, leukemias in adults and childhood, such as acute lymphoblastic leukemia (ALL), acute myeloid leukemia (AML), chronic myeloid leukemia (CML), myelodysplastic syndrome (MDS); solid tumors in childhood, such as neuroblastomas, Ewing sarcomas, and plasmocytomas/multiple myelomas; and other diseases, such as Morbus Hodgkin's disease (MHD) or Non-Hodgkin's lymphomas (NHL), and other inherited or acquired bone marrow failure syndromes such as aplastic anemia, Fanconi's anemia and Dyskeratosis congenita, Diamond Blackfan anemia, cMPL deficiency.

Optimal TBI requires individual treatment planning based on systematic dose measurements and CT-localization under treatment conditions, considering tissue inhomogeneities and individual body contours, careful performance of TBI with verification and control and documentation of all relevant treatment parameters. Methods known to those skilled in the art can be used to conduct the TBI in a method of the invention in view of the present disclosure. See, for example, Quast, J Med Phys. 2006, 31(1): 5-12, for a guideline on TBI, the entire content of which is incorporated herein by reference.

Chemotherapy

The term “chemotherapy,” as used herein, refers to a treatment of a disease that uses one or more chemical substances (chemotherapeutic agents). Preferably, chemotherapy can be a cancer treatment that uses one or more chemotherapeutic agents to kill cancer cells. Chemotherapy can be given with a curative intent, or it can aim to prolong life or to reduce symptoms. Chemotherapeutic agent, also referred to as chemotherapeutic compound, refers to any agent that can be used to treat a disease or disorder of a subject. Conventional chemotherapy uses non-specific cytotoxic drugs to inhibit cell division (mitosis).

Based on their principal mechanism of action, conventional chemotherapeutics can be broadly subdivided into: 1) alkylating agents; 2) antimetabolites; 3) topoisomerase inhibitors; 4) microtubular poisons: and 5) cytotoxic antibiotics.

Radiomimetic Chemotherapy

Radiomimetic chemotherapy is a type of chemotherapy that uses radiomimetic agents to kill cancer cells. As used herein, the term “radiomimetic agent” or “radiomimetic chemical agent” refers to a chemical agent that produces an effect similar to that of ionizing radiation when administered to a subject. Examples of such effect include DNA damage. Examples of radiomimetic agents include, but not limited to, ozone, peroxide, vesicants such as sulfur mustards and nitrogen mustards, alkylating agents (busulfan, melphalan, carmustine, cyclophosphamide, thiotepa, sarcolysine, chlorambucil), antimetabolite class of agents (fludarabine, clofrabine, cytarabine, 6-thioguanine), topoisomerase II inhibitor (etoposide), platinum-based agents, and cytotoxic antibiotics such as bleomycin and neocarzinostatin. Radiomimetic chemical agents such as those described herein can be administered locally to a subject to allow for a targeted application of the agent in a therapeutic manner.

Radiomimetic agents are similar to ionizing radiation in that they exert mutagenic and carcinogenic effects, cause acute and chronic degenerative changes in the bone marrow, intestinal mucosa, and genital organs in mammals, suppress the formation of antibodies, and impair oxidative phosphorylation and protein biosynthesis. Substances that have been isolated from irradiated organisms have an analogous effect; they are more frequently called radio-toxins.

Radiochemotherapy

Radiochemotherapy (RCTx, RT-CT), also referred to as chemoradiotherapy (CRT, CRTx) and chemoradiation, is the combination of radiotherapy and chemotherapy to treat cancer. Radiochemotherapy can be concurrent (together) or sequential (one after the other).

TPO Mimetic

As used herein, a “TPOm”, “TPO mimetic” or “thrombopoietin mimetic” refers to a compound comprising a peptide capable of binding to and activating a thrombopoietin receptor. Preferably, in a TPO mimetic useful for the invention, the peptide capable of binding to and activating a thrombopoietin receptor has no significant homology with thrombopoietin (TPO). The lack of homology with TPO reduces the potential for generation of TPO antibodies. Examples of such peptide useful in a TPO mimetic include, but are not limited to, those described in U.S. Publication Nos. 2003/0158116; 2005/0137133; 2006/0040866; 2006/0210542; 2007/0148091; 2008/0119384; U.S. Pat. Nos. 5,869,451; 7,091,311; 7,615,533; 8,227,422; International Patent Publications WO2007/021572; WO2007/094781; and WO2009/148954, the entire contents of which are incorporated herein by reference. More preferably, in a TPO mimetic useful for the invention, the peptide capable of binding to and activating a thrombopoietin receptor is covalently linked to a moiety that improves one or more properties of the peptide. By way of a non-limiting example, the moiety can be a hydrophilic polymer, including but not limited to polyethylene glycol (PEG), polypropylene glycol, polylactic acid and polyglycolic acid. The moiety can also be a polypeptide, such as a Fc region or an albumin.

In a preferred embodiment, a TPO mimetic useful for the invention comprises a peptide having the amino acid sequence of: IEGPTLRQXaaLAARYaa (SEQ ID NO:1), wherein Xaa is tryptophan (W) or β-(2-naphthyl)alanine (referred to herein as “2-Nal”), and Yaa is alanine (A) or sarcosine (referred herein as “Sar”). Preferably, the peptide of SEQ ID NO:1 is covalently linked to a PEG or fused to a Fc domain.

In some embodiments, a TPO mimetic useful for the invention comprises a peptide of SEQ ID NO:1 covalently linked to a PEG, preferably a PEG having an average molecular weight of between about 5,000 to about 30,000 Daltons. Preferably, the PEG is selected from the group consisting of monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM). The PEGylation of the peptide leads to a reduced clearance of the compound without loss of potency. See, e.g., U.S. Pat. No. 7,576,056, the entire contents of which are incorporated herein by reference.

In one preferred embodiment, a TPO mimetic useful for the invention is RWJ-800088 or a derivative thereof. As used herein, “RWJ-800088” refers to a 29-mer peptide having two identical 14-mers (SEQ ID NO:2) linked by a lysinamide residue as follows.

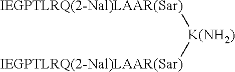

and having a methoxypoly(ethylene glycol) (MPEG) covalently linked to each N-terminal isoleucine, or a pharmaceutically acceptable salt or ester thereof. The RWJ-800088 is thus composed of two 14 amino acid peptide chains of SEQ ID NO:1, where Xaa is 2-Nal and Yaa is Sar, linked by lysinamide reside, and each N-terminal isoleucine is linked to a methoxy polyethylene glycol (MPEG) chain. Accordingly, RWJ-800088 has an abbreviated molecular structure of (MPEG-Ile-Glu-Gly-Pro-Thr-Leu-Arg-Gln-(2-Nal)-Leu-Ala-Ala-Arg-(Sar))2-Lys-NH2; wherein (2-Nal) is beta-(2-naphthyl)alanine, (Sar) is sarcosine and MPEG is methoxypoly(ethylene glycol), or a pharmaceutically acceptable salt or ester thereof. Preferably, the MPEG has an approximately 20,000 Dalton molecular weight or represents methoxypolyethylene glycol20000.

In one embodiment, RWJ-800088 has a molecular structure of formula (I), or a pharmaceutically acceptable salt or ester thereof:

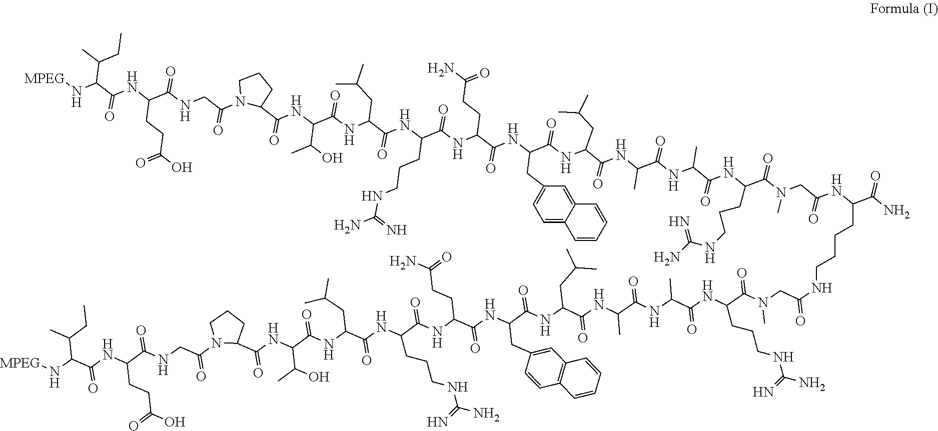

In a preferred embodiment, the MPEG in RWJ-800088 is methoxypolyethyleneglycol20000, and the RWJ-800088 has the full chemical name of: methoxypolyethyleneglycol20000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-Ne-(methoxypolyethyleneglycol20000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-)-Lysinamide, or a pharmaceutically acceptable salt or ester thereof. The molecular weight of the peptide without PEG is 3,295 Daltons and with two 20,000 Dalton MPEG chains is approximately 43,295 Daltons.

In some embodiments, a TPO mimetic useful for the invention comprises a peptide of SEQ ID NO:1 fused to a Fc domain. Fusing the peptide to a Fc domain can stabilize the peptide in vivo. See, e.g., U.S. Pat. No. 6,660,843, the entire contents of which are incorporated herein by reference.

In another preferred embodiment, a TPO mimetic useful for the invention is romiplostim. As used herein, “romiplostim” refers to fusion protein having a Fc domain linked to the N-terminal isoleucine of the peptide of SEQ ID NO:1, where Xaa is W and Yaa is A. In particular, romiplostim has the following amino acid sequence:

(SEQ ID NO: 4)
MDKTHTCPPCPAPELLGGPSVFLFPPKPKDTLMISRTEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGKGGGGGIEGPTLRQWLAARAGGGG
GGGGIEGPTLRQWLAARA,

It has the thrombopoietin receptor binding domain amino acid sequence of

| (SEQ ID NO: 3) | |
|---|---|
| | IEGPTLRQWLAARA. |

Dosage and Administration

The TPO mimetic can, for example, be administered as an active ingredient of a pharmaceutical composition in association with a pharmaceutical carrier or diluent. The TPO mimetics can be administered by oral, pulmonary, parental (intramuscular (IM), intraperitoneal (IP), intravenous (IV) or subcutaneous injection (SC)), inhalation (via a fine powder formulation), transdermal, nasal, vaginal, rectal, or sublingual routes of administration can be formulated in dosage forms appropriate for each route of administration. See, e.g., International Publication Nos. WO1993/25221 (Bernstein et al.) and WO1994/17784 (Pitt et al.), the relevant content of which is incorporated herein by reference.

Solid dosage forms for oral administration include capsules, tablets, pills, powders, and granules. In such solid dosage forms, the active peptide compound is admixed with at least one pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Preparations for parental administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through bacteria retaining filter, by incorporating sterilizing agents into the compositions, by irradiating the compositions, or by heating the compositions. They can also be manufactured using sterile water, or some other sterile injectable medium immediately before use.

Administration of the TPO mimetic is typically intramuscular, subcutaneous, or intravenous. However other modes of administration such as cutaneous, intradermal or nasal can be envisaged as well. Intramuscular administration of the TPO mimetic can be achieved by using a needle to inject a suspension of the TPO mimetic composition. An alternative is the use of a needleless injection device to administer the composition (using, e.g., Biojector™) or a freeze-dried powder of the TPO mimetic composition.

For intravenous, cutaneous or subcutaneous injection, or injection at the site of affliction, the TPO mimetic composition can be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

Compositions for rectal or vaginal administration are preferably suppositories which can contain, in addition to the active TPO mimetic, excipients such as cocoa butter or a suppository wax. Compositions for nasal or sublingual administration are also prepared with standard excipients well known in the art.

Typically, administration will have a therapeutic and/or prophylactic aim to mitigate vascular injury against a radiation therapy or a chemotherapy administered to the subject. In therapeutic applications, the TPO mimetic compositions are administered to a subject already dealing with vascular injury issues, and the TPO mimetic compositions are administered in an amount sufficient to cure or at least partially provide protective effects for the vasculature of the subject. In prophylactic applications, TPO mimetic compositions are administered to a subject susceptible to-or at risk of developing vascular injury conditions (e.g., a subject that will be exposed to a targeted radiation therapy). In each of these scenarios the amount of the TPO mimetic compositions will depend on the state of the subject (e.g., severity of the vasculature integrity, length of exposure to targeted radiation therapy) and the physical characteristics of the subject (e.g., height, weight, etc.).

The pharmaceutically acceptable compositions containing the TPO mimetic are administered to a subject, giving rise to protective effect on the vasculature of the subject. An amount of a composition sufficient to produce a protective effect on the vasculature of the subject is defined to be an “effective dose” or an “effective amount” of the composition.

In addition, administration of the TPO mimetic can enhance survival in a subject following TBI or local radiation. The dose of the TBI or local radiation can be lethal or supra-lethal.

The TPO mimetic can be administered once or multiple times before or after the radiation. Preferably, the TPO mimetic is RWJ-800088 or romiplostim. The dose of RWJ-800088 that provides maximal benefit in terms of survival is a single dose that produce a 2-4 elevation of platelets in healthy subjects. In the case of RWJ-800088 or romiplostim in humans this dose is 2.25-4 μg/kg administered SC, preferably 3 μg/kg, or the fixed or tiered dose equivalents based upon a typical body weight of the subject population, and produces 3× elevation in platelets in healthy subjects. In certain embodiments, a single dose of RWJ-800088 prior to lethal doses of whole-body irradiation is better than multiple doses from the perspective of survival.

The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. ed., 1980.

In certain embodiments, the TPO mimetic is administered to the subject at least about 10 minutes to at least about 420 minutes, at least about 10 minutes to at least about 300 minutes, at least about 10 minutes to at least about 180 minutes, at least about 10 minutes to at least about 60 minutes, at least about 20 minutes to at least about 420 minutes, at least about 20 minutes to at least about 300 minutes, at least about 20 minutes to at least about 180 minutes, at least about 20 minutes to at least about 60 minutes, at least about 40 minutes to at least about 420 minutes, at least about 40 minutes to at least about 300 minutes, at least about 40 minutes to at least about 180 minutes, at least about 40 minutes to at least about 60 minutes, at least about 60 minutes to at least about 420 minutes, at least about 60 minutes to at least about 300 minutes, at least about 60 minutes to at least about 180 minutes, at least about 60 minutes to at least about 120 minutes, at least about 60 minutes to at least about 90 minutes, at least about 80 minutes to at least about 420 minutes, at least about 80 minutes to at least about 300 minutes, at least about 80 minutes to at least about 180 minutes, at least about 80 minutes to at least about 120 minutes, at least about 100 minutes to at least about 420 minutes, at least about 100 minutes to at least about 300 minutes, at least about 100 minutes to at least about 180 minutes, at least about 100 minutes to at least about 150 minutes, at least about 120 minutes to at least about 420 minutes, at least about 120 minutes to at least about 300 minutes, at least about 120 minutes to at least about 180 minutes, at least about 140 minutes to at least about 420 minutes, at least about 140 minutes to at least about 300 minutes, at least about 140 minutes to at least about 180 minutes, at least about 160 minutes to at least about 420 minutes, at least about 160 minutes to at least about 300 minutes, at least about 160 minutes to at least about 180 minutes, at least about 180 minutes to at least about 420 minutes, at least about 180 minutes to at least about 300, or any amount in between after the subject is treated with the radiation or chemotherapy. In certain embodiments, the TPO mimetic is administered at least about 10, at least about 20, at least about 40, at least about 60, at least about 80, at least about 100, at least about 120, at least about 140, at least about 160, at least about 180, at least about 200, at least about 220, at least about 240, at least about 260, at least about 280, at least about 300, at least about 320, or at least about 340 minutes after the subject is treated with the radiation or chemotherapy. In certain embodiments, the TPO mimetic is administered at least about 8, at least about 10, at least about 12, at least about 14, at least about 16, at least about 18, at least about 20, at least about 22, or at least about 24 hours after the subject is treated with the radiation or chemotherapy. In certain embodiments, the TPO mimetic is administered no later than about 10, about 20, about 40, about 60, about 80, about 100, about 120, about 140, about 160, about 180, about 200, about 220, about 240, about 260, about 280, about 300, about 320, about 340, about 360, or about 420 minutes after the subject is treated with the radiation or chemotherapy. In certain embodiments, the TPO mimetic is administered no later than about 8, about 10, about 12, about 14, about 16, about 18, about 20, about 22, or about 24 hours after the subject is treated with the radiation or chemotherapy.

In certain embodiments, the TPO mimetic is administered to the subject at least about 10 minutes to at least about 240 minutes, at least about 10 minutes to at least about 180 minutes, at least about 10 minutes to at least about 60 minutes, at least about 20 minutes to at least about 240 minutes, at least about 20 minutes to at least about 180 minutes, at least about 20 minutes to at least about 60 minutes, at least about 40 minutes to at least about 240 minutes, at least about 40 minutes to at least about 180 minutes, at least about 40 minutes to at least about 60 minutes, at least about 60 minutes to at least about 240 minutes, at least about 60 minutes to at least about 180 minutes, at least about 60 minutes to at least about 120 minutes, at least about 60 minutes to at least about 90 minutes, at least about 80 minutes to at least about 240 minutes, at least about 80 minutes to at least about 180 minutes, at least about 80 minutes to at least about 120 minutes, at least about 100 minutes to at least about 240 minutes, at least about 100 minutes to at least about 180 minutes, at least about 100 minutes to at least about 150 minutes, at least about 120 minutes to at least about 240 minutes, at least about 120 minutes to at least about 180 minutes, at least about 140 minutes to at least about 240 minutes, at least about 140 minutes to at least about 180 minutes, at least about 160 minutes to at least about 240 minutes, at least about 160 minutes to at least about 180 minutes, at least about 180 minutes to at least about 240 minutes, at least about 180 minutes to at least about 200, or any amount in between prior to the subject being exposed to the radiation or chemotherapy. In certain embodiments, the TPO mimetic is administered at least about 240, at least about 220, at least about 200, at least about 180, at least about 160, at least about 140, at least about 120, at least about 100, at least about 80, at least about 60, at least about 40, at least about 30, at least about 20, or at least about 10 minutes prior to the subject being exposed to the radiation or chemotherapy. In certain embodiments, the TPO mimetic is administered at least about 24, at least about 22, at least about 20, at least about 18, at least about 16, at least about 14, at least about 12, at least about 10, at least about 8, at least about 6, at least about 4, or at least about 2 hours prior to the subject being exposed to the radiation or chemotherapy. In certain embodiments, the TPO mimetic is administered no later than about 240, about 220, about 200, about 180, about 160, about 140, about 120, about 100, about 80, about 60, about 40, about 30, about 20, or about 10 minutes prior to the subject being exposed to the radiation or chemotherapy. In certain embodiments, the TPO mimetic is administered no later than about 24, about 22, about 20, about 18, about 16, about 14, about 12, about 10, about 8, about 6, about 4, or about 2 hours prior to the subject being exposed to the radiation or chemotherapy.

In certain embodiments, the subject is administered a single dose of the effective amount of the TPO mimetic. In certain embodiments, the subject is administered multiple doses of the effective amount of the TPO mimetic

In certain embodiments, the effective amount of the TPO mimetic is about 0.1 μg to about 5 μg/kg, about 0.1 μg to about 4 μg/kg, about 0.1 μg to about 3 μg/kg, about 0.1 μg to about 2 μg/kg, about 0.1 μg to about 1 μg/kg, about 0.1 μg to about 0.5 μg/kg, about 0.1 μg to about 0.3 μg/kg, about 0.1 μg to about 0.2 μg/kg, about 0.5 μg to about 5 μg/kg, about 0.5 μg to about 4 μg/kg, about 0.5 μg to about 3 μg/kg, about 0.5 μg to about 2 μg/kg, about 0.5 μg to about 1 μg/kg, about 1 μg to about 5 μg/kg, about 1 μg to about 4 μg/kg, about 1 μg to about 3 μg/kg, about 1 μg to about 2 μg/kg, about 2 μg to about 5 μg/kg, about 2 μg to about 4 μg/kg, about 2 μg to about 3 μg/kg, about 3 μg to about 5 μg/kg, about 3 μg to about 4 μg/kg, or any amount in between, of body weight of the human subject, or the fixed or tiered dose equivalents based upon a typical body weight of the subject population. In preferred embodiments, the effective amount of the TPO mimetic is about 1, 1.25, 1.5, 1.75, 2, 2.25, 2.5, 2.75, 3, 3.25, 3.5, 3.75, 4, 4.25, 4.5, 4.75 or 5 μg/kg of body weight of the human subject, or the fixed or tiered dose equivalents based upon a typical body weight of the subject population. In preferred embodiments, the effective amount of the TPO mimetic is about 3 μg/kg of body weight of the human subject, or the fixed or tiered dose equivalents based upon a typical body weight of the subject population. An effective amount of the TPO mimetic can vary based on the species of the subject to be treated. In certain embodiments, wherein the subject is a mouse, the effective amount of the TPO mimetic is about 100 μg to about 5000 μg/kg, or any amount in between, of body weight of the subject. In certain embodiments, wherein the subject is a rat, the effective amount of the TPO mimetic is about 1000 μg to about 50,000 μg/kg, or any amount in between, of body weight of the subject. In certain embodiments, wherein the subject is a dog or a monkey, the effective amount of the TPO mimetic is about 10,000 μg to about 500,000 μg/kg, or any amount in between, of body weight of the subject.

Following production of the TPO mimetic and optional formulation of the TPO mimetic into compositions, the compositions can be administered to an individual, particularly human or other primate. Administration can be to humans, or another mammal, e.g., mouse, rat, hamster, guinea pig, rabbit, sheep, goat, pig, horse, cow, donkey, monkey, dog or cat. Delivery to a non-human mammal need not be for a therapeutic purpose, but can be for use in an experimental context, for instance in investigation of mechanisms of protecting vascular integrity due to administration of the TPO mimetic.

The TPO mimetic compositions can, if desired, be presented in a kit, pack or dispenser, which can contain one or more-unit dosage forms containing the active ingredient. The kit, for example, can comprise metal or plastic foil, such as a blister pack. The kit, pack, or dispenser can be accompanied by instructions for administration.

The TPO mimetic compositions of the invention can be administered alone or in combination with other treatments, either simultaneously or sequentially dependent upon the condition to be treated.

### Embodiments

The invention provides also the following non-limiting embodiments.

Embodiment 1(a) is a method of mitigating vascular injury in a human subject treated with a radiation therapy or a chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(b) is a method of promoting organ and/or hematopoietic recovery in a human subject treated with a radiation therapy or a chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(c) is a method of enhancing survival in a human subject treated with a radiation therapy or a chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(d) is a method of protecting against organ and hematopoietic injury in a human subject treated with a radiation therapy or a chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(e) is a method of enhancing or accelerating vascular recovery in a human subject treated with a radiation therapy or a chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(f) is a method of minimizing effects of a radiation therapy or a chemotherapy on blood cells and/or bone marrow in a human subject treated with the radiation therapy or the chemotherapy, the method comprising administering to the human subject in need thereof an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight of the subject, or the fixed or tiered dose equivalents based upon a typical body weight of the subject population.

Embodiment 1(g) is a method of treating a human subject in need of eradication of malignant cells and/or suppression of immune system, comprising:

a. treating the human subject with at least one of a radiation therapy and a radiomimetic chemotherapy, and

b. subcutaneously administering to the human subject an effective amount of a thrombopoietin (TPO) mimetic, wherein the TPO mimetic comprises the amino acid sequence of SEQ ID NO:1, and the effective amount comprises 0.1 microgram (μg) to 6 μg of the TPO mimetic per kilograms (kg) body weight, or the fixed or tiered dose equivalents based upon a typical body weight of the subject population.

Embodiment 2 is the method of any one of embodiments 1(a) to 1(g), wherein the TPO mimetic is administered to the subject within about 32 hours prior to and about 24 hours after the subject being treated with the at least one of a radiation therapy and a radiomimetic chemotherapy.

Embodiment 2(a) is the method of embodiment 2, wherein the TPO mimetic is administered to the subject about 0 minute to about 24 hours after the subject is treated with the radiation therapy or the chemotherapy.

Embodiment 2(b) is the method of embodiment 2 (a), wherein the TPO mimetic is administered to the subject about 10 minutes to about 20 hours after the subject being exposed to the radiation therapy or the chemotherapy.

Embodiment 2(c) is the method of embodiment 2 (a), wherein the TPO mimetic is administered to the subject about 10 minutes to about 16 hours after the subject being exposed to the radiation therapy or the chemotherapy.

Embodiment 2(d) is the method of embodiment 2 (a), wherein the TPO mimetic is administered to the subject about 10 minutes to about 12 hours after the subject being exposed to the radiation therapy or the chemotherapy.

Embodiment 2(e) is the method of embodiment 2 (a), wherein the TPO mimetic is administered to the subject about 10 minutes to about 8 hours after the subject being exposed to the radiation therapy or the chemotherapy.

Embodiment 2(f) is the method of embodiment 2 (a), wherein the TPO mimetic is administered to the subject about 10 minutes to about 4 hours after the subject being exposed to the radiation therapy or the chemotherapy.

Embodiment 2(g) is the method of embodiment 2 (a), wherein the TPO mimetic is administered to the subject about 0 minute, 10 minutes, 30 minutes, 1 hour, 1.5 hours, 2 hours, 2.5 hours, 3 hours, 3.5 hours, 4 hours, or any time in between, after the subject being exposed to the radiation therapy or the chemotherapy.

Embodiment 2 (h) is the method of embodiment 2, wherein the TPO mimetic is administered to the subject about 0 minute to about 32 hours before the subject is treated with the radiation therapy or the chemotherapy.

Embodiment 2(i) is the method of embodiment 2 (h), wherein the TPO mimetic is administered to the subject about 10 minutes to about 28 hours before the subject being exposed to the radiation therapy or the chemotherapy.

Embodiment 2(j) is the method of embodiment 2 (h), wherein the TPO mimetic is administered to the subject about 10 minutes to about 24 hours before the subject being exposed to the radiation therapy or the chemotherapy.

Embodiment 2(k) is the method of embodiment 2 (h), wherein the TPO mimetic is administered to the subject about 10 minutes to about 20 hours before the subject being exposed to the radiation therapy or the chemotherapy.

Embodiment 2(l) is the method of embodiment 2 (h), wherein the TPO mimetic is administered to the subject about 10 minutes to about 16 hours before the subject being exposed to the radiation therapy or the chemotherapy.

Embodiment 2(m) is the method of embodiment 2 (h), wherein the TPO mimetic is administered to the subject about 10 minutes to about 12 hours before the subject being exposed to the radiation therapy or the chemotherapy.

Embodiment 2(n) is the method of embodiment 2 (h), wherein the TPO mimetic is administered to the subject about 10 minutes to about 8 hours before the subject being exposed to the radiation therapy or the chemotherapy.

Embodiment 2(o) is the method of embodiment 2 (h), wherein the TPO mimetic is administered to the subject about 10 minutes to about 4 hours before the subject being exposed to the radiation therapy or the chemotherapy.

Embodiment 2(p) is the method of embodiment 2 (h), wherein the TPO mimetic is administered to the subject about 0 minute, 10 minutes, 30 minutes, 1 hour, 1.5 hours, 2 hours, 2.5 hours, 3 hours, 3.5 hours, 4 hours, or any time in between, before the subject being exposed to the radiation therapy or the chemotherapy.

Embodiment 3 (a) is a method of mitigating vascular injury in a subject treated with a radiation therapy or a chemotherapy, the method comprising administering to the subject in need thereof an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 3 (b) is a method of promoting organ and/or hematopoietic recovery in a subject treated with a radiation therapy or a chemotherapy, the method comprising administering to the subject in need thereof an effective amount of a thrombopoietin (TPO) mimetic, and the TPO mimetic is administered to the subject within about 32 hours prior to the subject being treated with the at least one of the radiation therapy and the radiomimetic chemotherapy.

Embodiment 3(c) is a method of enhancing survival in a subject treated with a radiation therapy or a chemotherapy, the method comprising administering to the subject in need thereof an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 3(d) is a method of protecting against organ and hematopoietic injury in a subject treated with a radiation therapy or a chemotherapy, the method comprising administering to the subject in need thereof an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 3(e) is a method of enhancing or accelerating vascular recovery in a subject treated with a radiation therapy or a chemotherapy, the method comprising administering to the subject in need thereof an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 3(f) is a method of minimizing effect of a radiation therapy or a chemotherapy on blood cells and/or bone marrow in a subject treated with the radiation therapy or the chemotherapy, the method comprising administering to the subject in need thereof an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 3(g) is a method of treating a subject in need of eradication of malignant cells and/or suppression of immune system, comprising:

a. treating the subject with at least one of a radiation therapy and a radiomimetic chemotherapy, and

b. subcutaneously administering to the subject an effective amount of a thrombopoietin (TPO) mimetic, wherein the TPO mimetic comprises the amino acid sequence of SEQ ID NO:1, and the TPO mimetic is administered to the subject within about 32 hours prior to the subject being treated with the at least one of the radiation therapy and the radiomimetic chemotherapy.

Embodiment 4 is the method of any one of embodiment 3 (a) to 3(g), wherein the effective amount of TPO mimetic is administered to the subject subcutaneously.

Embodiment 4 (a) is the method of embodiment 4, wherein the subject is a human being, and the effective amount of the TPO mimetic is about 0.1 microgram (μg) to about 6 μg of the TPO mimetic per kilograms (kg) body weight of the subject, or the fixed or tiered dose equivalents based upon a typical body weight of the subject population.

Embodiment 4 (b) is the method of embodiment 4, wherein the subject is a mouse, and the effective amount of the TPO mimetic is about 100 μg to about 5000 μg/kg body weight of the subject.

Embodiment 4 (c) is the method of embodiment 4, wherein the subject is a rat, and the effective amount of the TPO mimetic is about 1000 μg to about 50,000 μg/kg body weight of the subject.

Embodiment 4 (d) is the method of embodiment 4, wherein the subject is a dog or monkey, and the effective amount of the TPO mimetic is about 10,000 μg to about 50,000 μg/kg body weight of the subject.

Embodiment 4 (e) is the method of any one of embodiments 3 (a) to 4(d), wherein the TPO mimetic is administered to the subject about 10 minutes to about 28 hours before the subject being exposed to the radiation therapy or the chemotherapy.

Embodiment 4 (f) is the method of embodiment 4 (e), wherein the TPO mimetic is administered to the subject about 10 minutes to about 24 hours before the subject being exposed to the radiation therapy or the chemotherapy.

Embodiment 4 (g) is the method of embodiment 4 (e), wherein the TPO mimetic is administered to the subject about 10 minutes to about 20 hours before the subject being exposed to the radiation therapy or the chemotherapy.

Embodiment 4 (h) is the method of embodiment 4 (e), wherein the TPO mimetic is administered to the subject about 10 minutes to about 16 hours before the subject being exposed to the radiation therapy or the chemotherapy.

Embodiment 4 (i) is the method of embodiment 4 (e), wherein the TPO mimetic is administered to the subject about 10 minutes to about 12 hours before the subject being exposed to the radiation therapy or the chemotherapy.

Embodiment 4 (j) is the method of embodiment 4 (e), wherein the TPO mimetic is administered to the subject about 10 minutes to about 8 hours before the subject being exposed to the radiation therapy or the chemotherapy.

Embodiment 4 (k) is the method of embodiment 4 (e), wherein the TPO mimetic is administered to the subject about 10 minutes to about 4 hours before the subject being exposed to the radiation therapy or the chemotherapy.

Embodiment 4 (l) is the method of embodiment 4 (e), wherein the TPO mimetic is administered to the subject about 0 minute, 10 minutes, 30 minutes, 1 hour, 1.5 hours, 2 hours, 2.5 hours, 3 hours, 3.5 hours, 4 hours, or any time in between, before the subject being exposed to the radiation therapy or the chemotherapy

Embodiment 5 is the method of any one of embodiments 1 to 4(1), wherein the peptide has the amino acid sequence of SEQ ID NO:2.

Embodiment 5 (a) is the method of embodiment 5, wherein the TPO mimetic further comprises a hydrophilic polymer covalently linked to the peptide.

Embodiment 5 (b) is the method of embodiment 5 (a), wherein the hydrophilic polymer is any one of: i) polyethylene glycol (PEG), ii) polypropylene glycol, iii) polylactic acid, or iv) polyglycolic acid.

Embodiment 5 (c) is the method of embodiment 5 (b), wherein the hydrophilic polymer is PEG.

Embodiment 5 (d) is the method of embodiment 5 (c), wherein the PEG is any one of monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), or monomethoxypolyethylene glycol-imidazolyl-carbonyl (MePEG-IM).

Embodiment 5 (e) is the method of embodiment 5 (d), wherein the PEG is methoxypoly(ethylene glycol) (MPEG).

Embodiment 5 (f) is the method of embodiment 5 (e), wherein the TPO mimetic is RWJ-800088 having a molecular structure of formula (I), or a pharmaceutically acceptable salt or ester thereof:

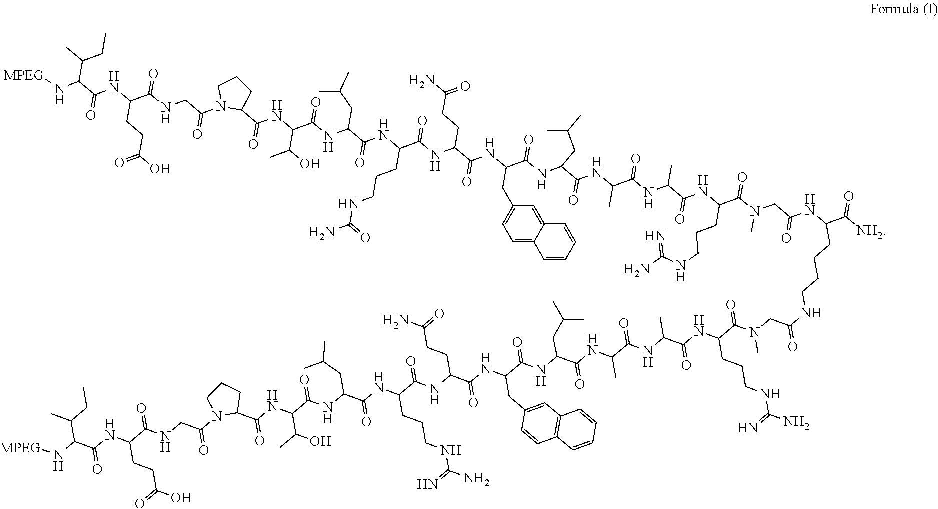

Embodiment 5 (g) is the method of embodiment 5 (f), wherein the MPEG in the RWJ-800088 is methoxypolyethylene glycol20000.

Embodiment 6 is the method of any one of embodiments 1 to 4(d), wherein the peptide has the amino acid sequence of SEQ ID NO:3.

Embodiment 6 (a) is the method of embodiment 6, wherein the peptide is fused to a polypeptide.

Embodiment 6 (b) is the method of embodiment 6 (a), wherein the polypeptide is a Fc domain.

Embodiment 6 (c) is the method of embodiment 6 (b), wherein the TPO mimetic is romiplostim.

Embodiment 6 (d) is the method of embodiment 6 (c), wherein romiplostim comprises the amino acid sequence of SEQ ID NO:4.

Embodiment 7 is the method of any of embodiments 1 to 6 (d), wherein the subject is treated for mitigation of toxicity and enhancement of survival for Acute Radiation Syndrome or radiation and/or chemotherapy used in bone marrow transplant conditioning.

Embodiment 8 is the method of any embodiments 1 to 6 (d), wherein the subject is treated with a radiation therapy, wherein the radiation therapy is total body irradiation.

Embodiment 8 (a) is the method of embodiment 8, wherein the subject is treated with the total body irradiation prior to bone marrow transplant.

Embodiment 8 (b) is the method of embodiment 8 or 8 (a), wherein the subject is treated for a leukemia, preferably an acute lymphoblastic leukemia (ALL), acute myeloid leukemia (AML), chronic myeloid leukemia (CML) or myelodysplastic syndrome (MDS).

Embodiment 8 (c) is the method of embodiment 8 or 8 (a), wherein the subject is treated for a solid tumor, preferably neuroblastomas, Ewing sarcomas, plasmocytomas, or multiple myelomas, more preferably, the subject is a child.

Embodiment 8 (d) is the method of embodiment 8 or 8 (a), wherein the subject is treated for Morbus Hodgkin's disease (MHD) or Non-Hodgkin's lymphomas (NHL).

Embodiment 9 is the method of any one of embodiments 1 to 6 (d), wherein the subject is treated with a chemotherapy.

Embodiment 9 (a) is the method of embodiment 9, wherein the subject treated with the chemotherapy is being treated for cancer.

Embodiment 9 (b) is the method of embodiment 9 (a), wherein the cancer is selected from the group consisting of prostate cancer, head and neck cancer, hepatocellular carcinoma, colon cancer, lung cancer, melanoma, pancreatic, breast cancer, an acute lymphoblastic leukemia (ALL), acute myeloid leukemia (AML), chronic myeloid leukemia (CML), myelodysplastic syndrome (MDS), neuroblastomas, Ewing sarcomas, plasmocytomas, multiple myelomas, Morbus Hodgkin's disease (MHD) and Non-Hodgkin's lymphomas (NHL).

Embodiment 9 (c) is the method of any one of embodiments 7 to 8 (d), wherein the subject is treated with the radiation therapy and the chemotherapy.

Embodiment 9 (d) is the method of any one of embodiment 9 to 9 (c), wherein the chemotherapy is a radiomimetic chemotherapy.

Embodiment 9 (e) is the method of embodiment 9 (d), wherein the radiomimetic chemotherapy is selected from the group consisting of ozone, peroxide, vesicants (such as sulfur mustards and nitrogen mustards), alkylating agents (such as sarcolysine, busulfan, chlorambucil), platinum-based agents, and cytotoxic antibiotics (such as bleomycin and neocarzinostatin).

Embodiment 9 (f) is the method of embodiment 9 (e), wherein the radiomimetic chemotherapy is cyclophosphamide, busulfan, fludarabine, melphalan, thiotepa, cytarabine and clofarabine, carmustine, etoposide, cytarabine and melphalan, Rituximab, ifosfamide, etoposide, or a platinum-based agent selected from the group consisting of cisplatin, carboplatin, oxaliplatin, and nedaplatin.

Embodiment 10 is the method of any one of embodiments 1 to 9 (f), wherein the subject is administered a single dose of the effective amount of the TPO mimetic.

Embodiment 11 is the method of any one of embodiments 1 to 9 (f), wherein the subject is administered more than one dose of the effective amount of the TPO mimetic.

Embodiment 12 is the method of any one of embodiments 1 to 11, wherein the subject is a human being, and the effective amount of the TPO mimetic is about 0.5 μg to about 5 μg/kg of body weight of the subject, or the fixed or tiered dose equivalents based upon a typical body weight of the subject population.

Embodiment 12 (a) is the method of any one of embodiments 1 to 11, wherein the effective amount of the TPO mimetic is about 1 μg to about 4 μg/kg of body weight of the subject, or the fixed or tiered dose equivalents based upon a typical body weight of the subject population.

Embodiment 12 (b) is the method of any one of embodiments 1 to 11, wherein the effective amount of the TPO mimetic is about 2 μg to about 4 μg/kg of body weight of the subject, or the fixed or tiered dose equivalents based upon a typical body weight of the subject population.

Embodiment 12 (c) is the method of any one of embodiments 1 to 11, wherein the effective amount of the TPO mimetic is about 2 μg/kg, 2.25 μg/kg, 2.5 μg/kg, 2.75 μg/kg, 3 μg/kg, 3.25 μg/kg, 3.5 μg/kg, 3.75 μg/kg, 4 μg/kg, or any amount in between, of body weight of the subject, or the fixed or tiered dose equivalents based upon a typical body weight of the subject population.

Embodiment 12 (d) is the method of any one of embodiments 1 to 11, wherein the effective amount of the TPO mimetic is about 3 μg/kg of body weight of the subject, or the fixed or tiered dose equivalents based upon a typical body weight of the subject population.

Embodiment 13 is a method of treating a cancer in a human subject in need of, comprising:

a. treating the human subject with at least one of a radiation therapy and a radiomimetic chemotherapy, and

b. subcutaneously administering to the human subject an effective amount of a thrombopoietin (TPO) mimetic comprising RWJ-800088, wherein the effective amount comprises 0.1 microgram (μg) to 6 μg of the TPO mimetic per kilograms (kg) body weight of the subject, or the fixed or tiered dose equivalents based upon a typical body weight of the subject population, and the TPO mimetic is administered to the subject about 32 hours before to about 24 hours after the subject is treated with the at least one of the radiation therapy and the radiomimetic chemotherapy.

Embodiment 14 is a method of treating a cancer in a human subject in need of, comprising:

a. treating the human subject with at least one of a radiation therapy and a radiomimetic chemotherapy, and

b. subcutaneously administering to the human subject an effective amount of a thrombopoietin (TPO) mimetic comprising romiplostim, wherein the effective amount comprises 0.1 microgram (μg) to 6 μg of the TPO mimetic per kilograms (kg) body weight of the subject, or the fixed or tiered dose equivalents based upon a typical body weight of the subject population, and the TPO mimetic is administered to the subject about 32 hours before to about 24 hours after the subject is treated with the at least one of the radiation therapy and the radiomimetic chemotherapy.

Embodiment 15 is the method of embodiment 13 or 14, wherein the subject is treated with a total body irradiation.

Embodiment 15 (a) is the method of embodiment 15, wherein the cancer is selected from the group consisting of leukemia, solid tumor, Morbus Hodgkin's disease (MHD), and Non-Hodgkin's lymphomas (NHL).

Embodiment 15(b) is the method of embodiment 15(a), wherein the leukemia is an acute lymphoblastic leukemia (ALL), acute myeloid leukemia (AML), chronic myeloid leukemia (CML) or myelodysplastic syndrome (MDS).

Embodiment 15 (c) is the method of embodiment 15(a), wherein the solid tumor is neuroblastomas, Ewing sarcomas, plasmocytomas, or multiple myelomas.

Embodiment 16 is the method of embodiment 13 or 14, wherein the subject is treated with a total body irradiation prior to a transplant.

Embodiment 16 (a) is the method of embodiment 16, wherein the transplant is a transplantation of at least one of haematopoietic stem cells, bone marrow stem cells and peripheral blood progenitor stem cells.

Embodiment 16 (b) is the method of embodiment 16 or 16 (a), wherein the cancer is selected from the group consisting of a leukemia.

Embodiment 16 (c) is the method of embodiment 16 (b), wherein the cancer is an acute lymphoblastic leukemia (ALL), acute myeloid leukemia (AML), chronic myeloid leukemia (CML) or myelodysplastic syndrome (MDS).

Embodiment 16 (d) is the method of embodiment 16 or 16 (a), wherein the subject is treated for a solid tumor.

Embodiment 16 (e) is the method of embodiment 16 (d), wherein the cancer is neuroblastomas, Ewing sarcomas, plasmocytomas, or multiple myelomas.

Embodiment 16 (f) is the method of embodiment 16 (e), wherein the subject is a child.

Embodiment 16 (g) is the method of embodiment 16 or 16 (a), wherein the subject is treated for Morbus Hodgkin's disease (MHD) or Non-Hodgkin's lymphomas (NHL).

Embodiment 17 (a) is the method of embodiment 13 or 14, wherein the subject is treated with a chemotherapy.

Embodiment 17 (b) is the method of embodiment 17 (a), wherein the cancer is selected from the group consisting of prostate cancer, head and neck cancer, hepatocellular carcinoma, colon cancer, lung cancer, melanoma, pancreatic, breast cancer, an acute lymphoblastic leukemia (ALL), acute myeloid leukemia (AML), chronic myeloid leukemia (CML), myelodysplastic syndrome (MDS), neuroblastomas, Ewing sarcomas, plasmocytomas, multiple myelomas, Morbus Hodgkin's disease (MHD) and Non-Hodgkin's lymphomas (NHL).

Embodiment 17 (c) is the method of any one of embodiments 15 to 16 (g), wherein the subject is further treated with a chemotherapy.

Embodiment 17 (d) is the method of any one of embodiment 17 (a) to 17 (c), wherein the chemotherapy is a radiomimetic chemotherapy.

Embodiment 17 (e) is the method of embodiment 17 (d), wherein the radiomimetic chemotherapy is selected from the group consisting of cyclophosphamide, busulfan, fludarabine, melphalan, thiotepa, cytarabine and clofarabine, carmustine, etoposide, cytarabine and melphalan, Rituximab, ifosfamide, etoposide, ozone, peroxide, vesicants (such as sulfur mustards and nitrogen mustards), chlorambucil, platinum-based agents, and cytotoxic antibiotics (such as bleomycin and neocarzinostatin).

Embodiment 17 (f) is the method of embodiment 17 (e), wherein the radiomimetic chemotherapy is a platinum-based agent selected from the group consisting of cisplatin, carboplatin, oxaliplatin, and nedaplatin.

Embodiment 18 is the method of any one of embodiments 13 to 17 (f), wherein the TPO mimetic is administered to the subject about 0 minute to about 32 hours prior to the subject being exposed to the radiation therapy or the chemotherapy.

Embodiment 18 (a) is the method of any one of embodiments 13 to 17 (f), wherein the TPO mimetic is administered to the subject about 10 minutes to about 24 hours prior to the subject being exposed to the radiation therapy or the chemotherapy.

Embodiment 18 (b) is the method of any one of embodiments 13 to 17 (f), wherein the TPO mimetic is administered to the subject about 10 minutes to about 20 hours prior to the subject being exposed to the radiation therapy or the chemotherapy.

Embodiment 18 (c) is the method of any one of embodiments 13 to 17 (f), wherein the TPO mimetic is administered to the subject about 10 minutes to about 16 hours prior to the subject being exposed to the radiation therapy or the chemotherapy.

Embodiment 18 (d) is the method of any one of embodiments 13 to 17 (f), wherein the TPO mimetic is administered to the subject about 10 minutes to about 12 hours prior to the subject being exposed to the radiation therapy or the chemotherapy.

Embodiment 18 (e) is the method of any one of embodiments 13 to 17 (f), wherein the TPO mimetic is administered to the subject about 10 minutes to about 8 hours prior to the subject being exposed to the radiation therapy or the chemotherapy.

Embodiment 18 (f) is the method of any one of embodiments 13 to 17 (f), wherein the TPO mimetic is administered to the subject about 10 minutes to about 4 hours prior to the subject being exposed to the radiation therapy or the chemotherapy.

Embodiment 18 (g) is the method of any one of embodiments 13 to 17 (f), wherein the TPO mimetic is administered to the subject about 10 minutes, 30 minutes, 1 hour, 1.5 hours, 2 hours, 2.5 hours, 3 hours, 3.5 hours, 4 hours or any time in between, prior to the subject being exposed to the radiation therapy or the chemotherapy.

Embodiment 19 is the method of any one of embodiments 13 to 17 (f), wherein the TPO mimetic is administered to the subject about 0 minute to about 24 hours after the subject is treated with the radiation therapy or the chemotherapy.

Embodiment 19 (b) is the method of any one of embodiments 13 to 17 (f), wherein the TPO mimetic is administered to the subject about 10 minutes to about 20 hours after the subject being exposed to the radiation therapy or the chemotherapy.

Embodiment 19 (c) is the method of any one of embodiments 13 to 17 (f), wherein the TPO mimetic is administered to the subject about 10 minutes to about 16 hours after the subject being exposed to the radiation therapy or the chemotherapy.

Embodiment 19 (d) is the method of any one of embodiments 13 to 17 (f), wherein the TPO mimetic is administered to the subject about 10 minutes to about 12 hours after the subject being exposed to the radiation therapy or the chemotherapy.

Embodiment 19 (e) is the method of any one of embodiments 13 to 17 (f), wherein the TPO mimetic is administered to the subject about 10 minutes to about 8 hours after the subject being exposed to the radiation therapy or the chemotherapy.

Embodiment 19 (f) is the method of any one of embodiments 13 to 17 (f), wherein the TPO mimetic is administered to the subject about 10 minutes to about 4 hours after the subject being exposed to the radiation therapy or the chemotherapy.

Embodiment 19 (g) is the method of any one of embodiments 13 to 17 (f), wherein the TPO mimetic is administered to the subject about 10 minutes, 30 minutes, 1 hour, 1.5 hours, 2 hours, 2.5 hours, 3 hours, 3.5 hours, 4 hours, or any time in between, after the subject being exposed to the radiation therapy or the chemotherapy.

Embodiment 20 is the method of any one of embodiments 13 to 19 (g), wherein the subject is administered a single dose of the effective amount of the TPO mimetic.

Embodiment 21 is the method of any one of embodiments 13 to 19 (g), wherein the subject is administered more than one dose of the effective amount of the TPO mimetic.

Embodiment 22 is the method of any one of embodiments 13 to 21, wherein the effective amount of the TPO mimetic is about 0.5 μg to about 5 μg/kg of body weight of the subject, or the fixed or tiered dose equivalents based upon a typical body weight of the subject population.

Embodiment 22 (a) is the method of any one of embodiments 13 to 21, wherein the effective amount of the TPO mimetic is about 1 μg to about 4 μg/kg of body weight of the subject, or the fixed or tiered dose equivalents based upon a typical body weight of the subject population.

Embodiment 22 (b) is the method of any one of embodiments 13 to 21, wherein the effective amount of the TPO mimetic is about 2 μg to about 4 μg/kg of body weight of the subject, or the fixed or tiered dose equivalents based upon a typical body weight of the subject population.

Embodiment 22 (c) is the method of any one of embodiments 13 to 21, wherein the effective amount of the TPO mimetic is about 2 μg/kg, 2.25 μg/kg, 2.5 μg/kg, 2.75 μg/kg, 3 μg/kg, 3.25 μg/kg, 3.5 μg/kg, 3.75 μg/kg, 4 μg/kg, or any amount in between, of body weight of the subject, or the fixed or tiered dose equivalents based upon a typical body weight of the subject population.

Embodiment 22 (d) is the method of any one of embodiments 13 to 21, wherein the effective amount of the TPO mimetic is about 3 μg/kg of body weight of the subject, or the fixed or tiered dose equivalents based upon a typical body weight of the subject population.

Embodiment 23 is a pharmaceutical composition comprising the effective amount of a thrombopoietin (TPO) mimetic for use in the method of any one of embodiments 1 to 22 (d).

Embodiment 24 is a kit for mitigating vascular injury in a human subject treated with a radiation therapy or a chemotherapy, comprising the pharmaceutical composition of embodiment 23, and at least one additional therapeutic agent or device for mitigating the vascular injury, optionally, the kit further comprising a tool for administering the TPO mimetic to the subject.

Embodiment 25 is a kit for protecting against organ and hematopoietic injury in a subject treated with a radiation therapy or a chemotherapy, comprising the pharmaceutical composition of embodiment 23, and at least one additional therapeutic agent or device for protecting against organ and hematopoietic injury, optionally, the kit further comprising a tool for administering the TPO mimetic to the subject.

Embodiment 26 is a kit for promoting functional organ and hematopoietic recovery in a subject treated with a radiation therapy or a chemotherapy, comprising the pharmaceutical composition of embodiment 23, and at least one additional therapeutic agent or device for promoting functional organ and hematopoietic recovery, optionally, the kit further comprising a tool for administering the TPO mimetic to the subject.

Embodiment 27 is a kit for enhancing or accelerating vascular recovery in a subject treated with a radiation therapy or a chemotherapy, comprising the pharmaceutical composition of embodiment 23, and at least one additional therapeutic agent or device for enhancing or accelerating vascular recovery, optionally, the kit further comprising a tool for administering the TPO mimetic to the subject.

Embodiment 28 is a kit for enhancing survival in a subject treated with total body irradiation, comprising the pharmaceutical composition of embodiment 23, and at least one additional therapeutic agent or device for enhancing the survival, optionally, the kit further comprising a tool for administering the TPO mimetic to the subject.

Embodiment 29 is a kit for minimizing effects of total body irradiation on blood cells and/or bone marrow in a subject treated with the total body irradiation, comprising the pharmaceutical composition of embodiment 23, and at least one additional therapeutic agent or device for enhancing the survival, optionally, the kit further comprising a tool for administering the TPO mimetic to the subject.

### EXAMPLES

### Example 1: RWJ-800088 Protects Against Chemotherapy Induced Mortality and the Development of Microangiopathic Events in Mice

Materials and Methods: Increasing doses of carboplatin (i.e., 60, 70, or 80 mg/kg, i.p.) were administered on 2 consecutive days (Day 1 and Day 0) to male BALB/c mice (N=3 per group). Approximately 1 hour after carboplatin treatment on Day 0, sets of mice were treated with vehicle or RWJ-800088 (100 μg/kg, i.v.).

On Day 15, surviving mice were euthanized and blood samples were collected for the assessment of platelet and red blood cell (RBC) parameters. The brains of the mice were also isolated and preserved in 10% buffered formalin for immunohistochemical staining using anti-fibrinogen antibodies. Control mice were processed in a similar manner.

Results

Effects of RWJ-800088 on Carboplatin Induced Thrombocytopenia and Anemia

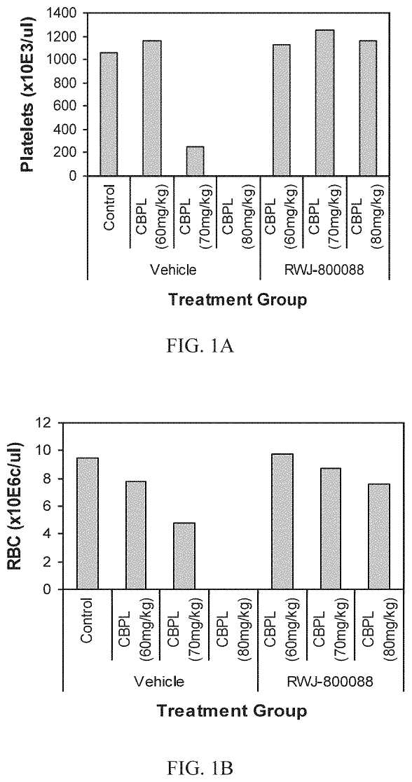

Treatment of the mice with increasing amounts of carboplatin induced a marked, dose-dependent decrease in platelet and RBC counts as observed on Day 15 (FIGS. 1A-B). In addition, all of the mice receiving 80 mg/kg carboplatin (alone) for 2 consecutive days were either found dead or were euthanized (due to profound moribund condition) prior to study termination. Co-treatment with RWJ-800088 prevented the observed decreases in platelet and RBC counts. Furthermore, none of the mice treated with RWJ-800088 died or required euthanization.

Effects of RWJ-800088 on Development of Carboplatin Induced Microangiopathic Events in the Brains of Mice

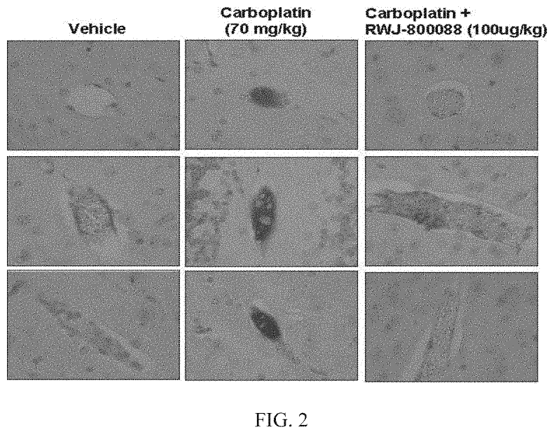

Immunohistochemical analysis of fixed brain sections isolated from mice treated with carboplatin (70 mg/kg) alone showed numerous fibrinogen-positive microvessels (FIG. 2). Many of the vessels were totally occluded by fibrinogen clots and some of the brain tissue exhibited signs of internal hemorrhage and edema. In marked contrast, histological sections from mice co-treated with RWJ-800088 appeared normal with the intensity of fibrinogen staining similar to vessels in the brains of control mice. Vessels, which were totally occluded with fibrinogen-positive clots, were a very infrequent observation and none of the brains isolated from mice co-treated with RWJ-800088 exhibited signs of hemorrhage or edema.

These results indicate that RWJ-800088 can prevent hematopoietic failure such as thrombocytopenia, anemia, and mortality induced by chemotherapy in mice. The histological findings suggest that the micro-clots that form in the small blood vessels following chemotherapy can contribute to the development of thrombocytopenia (due to platelet deposition) and anemia (due to microhemorrhage and RBC lysis). Furthermore, the ability of RWJ-800088 to prevent the development of the microangiopathic events can contribute to the ability of this agent to affect the development of chemotherapy-induced thrombocytopenia and anemia.

The ability of RWJ-800088 to prevent internal hemorrhage and edema in the brain is further evidence of its effects on preventing vascular damage and leakage.

### Example 2: Impact of Timing of Administration and Dose of RWJ-800088 on Survival, Hematopoietic and Vascular Injury Following Exposure to Lethal and Supralethal Doses of Irradiation in Mice

Mouse Whole Body Irradiation Studies

Animals: Male CD2F1 mice (8-10 weeks old) and C3H/HeN mice were purchased from Envigo (Indianapolis, Ind.) and male C57BL/6 mice (8-10 weeks old) were purchased from Jackson Laboratories (Bar Harbor, Me.). The mice were housed in the Armed Forces Radiobiology Research Institute's (AFRRI) vivarium accredited by the Association for Assessment and Accreditation of Laboratory Animal Care-International Experimental animals were identified by tail tattoo and housed 4 per box in sterile polycarbonate boxes with filter covers (Microisolator, Lab Products Inc., Seaford, Del.) and autoclaved hardwood chip bedding. The animals received Harlan Teklad Rodent Diet 8604 and acidified water (pH 2.5-3.0) ad libitum and were acclimatized for 1-2 weeks before the start of each study. The animal rooms were maintained at 21±2° C. and 50±10% relative humidity with 10-15 cycles of fresh air hourly and a 12:12 h light:dark cycle. All procedures pertaining to animals were reviewed and approved by the AFRRI Institutional Animal Care and Use Committee (IACUC) using the principles outlined in the National Research Council's Guide for the Care and Use of Laboratory Animals.

RWJ-800088 synthesis and administration: RWJ-800088 was in a powder form and it was formulated in normal sterile saline (0.9% NaCl) before use and protected from light. Either drug or its vehicle was injected subcutaneously (SC) at the nape of the neck, pre-TBI at the time indicated for each study prior to TBI.

Total Body Irradiation (TBI). Mice were irradiated bilaterally in the Cobalt-60 gamma-irradiation facility at the AFRRI. These animals were placed in well-ventilated plexiglass chambers made specifically for irradiating mice. An alanine/Electron Spin Resonance (ESR) dosimetry system (American Society for Testing and Material Standard E 1607) was used to measure the dose rates in the cores of acrylic phantoms (3 inches long and 1 inch in diameter) located in all empty slots of the exposure rack in the plexiglass chamber. ESR signals were measured with a calibration curve based on standard calibration dosimeters provided by the National Institute of Standard and Technology (NIST, Gaithersburg, Md.). The calibration curve was verified by inter-comparison with the National Physical Laboratory (NPL) in the United Kingdom. The corrections applied to the measured dose rates in phantoms were for decay of the Co-60 source and for a small difference in mass-energy absorption coefficients for water and soft tissue at the Co-60 energy level. Kaplan-Meier survival curves were plotted using GraphPad software; and trend in survival is compared between vehicle and drug-treated group.

Housing and care of animals after irradiation: After irradiation animals were returned to their cages and monitored three to four times daily (early morning, late morning, late afternoon and evening). Any sick animals were monitored closely, and their health was scored in accordance with pre-defined criteria described and approved in the IACUC protocol. The animals who reached the predetermined health score were euthanized according to American Veterinary Medical Association (AVMA) guidelines.

Prophylactic survival efficacy with a single dose of RWJ-800088 in CD2F1: The initial survival study consisted of testing one drug dose of RWJ-800088 (0.3 mg/kg), one route of administration (SC), and one radiation dose (LD70/30 [70% mortality over a 30 day period]=9.25 Gy). CD2F1 male mice were weighed (animals outside±10% of the mean weight were excluded), and randomized into groups of 4 animals per box. There were 24 animals per treatment group (6 boxes) for RWJ-800088 and its vehicle. The mice received SC administration of either RWJ-800088 or saline (the vehicle) at 24 h prior to TBI. After radiation exposure, the mice were monitored daily (three times a day when necessary) for 30 days and surviving animals were euthanized at the completion of the observational period. Survival data was plotted as Kaplan-Meier plots and statistical significance of the survival differences was determined by Log-rank test using GraphPad Prism 7 software.

Dose and time optimization study with RWJ-800088 in CD2F1 male mice: Five doses of RWJ-800088 (0.1, 0.3, 1.0, 2.0, 3.0 mg/kg) were selected to determine the optimum dose of single administration of RWJ-800088 to achieve maximum efficacy at 24 hours pre-TBI in CD2F1 mice. Mice (n=24/group) were administered SC either RWJ-800088 (0.1, 0.3, 1.0, and 3.0 mg/kg) or saline 24 h before exposure at 9.75 Gy (˜LD90/30). To determine the optimum prophylactic dose of RWJ-800088 to achieve maximum efficacy, these doses of RWJ-800088 were tested at two supralethal doses (10.5 and 11 Gy) of gamma-radiation as stated above. Animals were monitored for 30-days in the same way as described previously for the survival studies.

A time optimization study was performed by selecting different time points (2, 12 and 24 h pre-TBI). Four groups including saline and three RWJ-800088 (0.3 mg/kg) treatment groups were used in this study. Each group (N=24/group) was administered either RWJ-800088 at the specific time points or saline (only 24 h pre-TBI) before exposure to 9.75 Gy (˜-LD90/30). Animals were monitored for 30-days as described above.

Hematological recovery with RWJ-800088: To study the effects of prophylactic administration of RWJ-800088 on the recovery from hematopoietic injury following TBI, CD2F1 mice (n=10 per group) were treated with either a single dose of RWJ-800088 (0.3 mg/kg) or its vehicle (saline) 24 hours pre-TBI at a non-lethal dose of 7.0 Gy. This dose allows the animals to recover completely from the radiation-induced hematopoietic injury. In addition, a group of sham irradiated mice were given either the drug or saline. Blood collection was performed using a 23 G needle from the submandibular vein after anesthetizing the mice with isoflurane (Hospira Inc., Lake Forest, Ill.) at 2 hours and on days 1, 3, 7, 10, 14, 21, and 30 post-TBI. All mice were allowed to recover fully from anesthesia and monitored closely for any signs of a post-anesthesia reaction or bleeding at the collection site before returned to group housed cages. Approximately 20 μL of blood was collected in EDTA tubes and was continually rotated until CBC/differential analysis was completed using a HESKA Element HT™ 5 Analyzer system (HESKA Corporation, Loveland, Colo.). This CBC/differential analysis included white blood cells (WBC) counts, absolute neutrophil counts (Liem-Moolenaar, Clin Pharmacol Ther. 2008 October; 84(4):481-7), monocytes (MON), lymphocytes (LYM), red blood cells (RBC), hematocrits (HCT), and platelets (PLT) counts.

Harvesting blood and tissues for various molecular assays: RWJ-800088 (0.3 mg/kg) or saline (n=6 per group) was administered SC 24 hour prior to irradiation. The experimental animals received either 0 or 7 Gy radiation (non-lethal dose) in the AFRRI Co-60 gamma radiation facility. Blood was collected from the inferior vena cava under anesthesia on days 0 (2 h post-TBI), 1, 2, 3, 7, 15, and 30 after 7 Gy exposure or from unirradiated mice (at the same time points) followed by euthanasia. The femurs and sterna were then collected and processed as described below.

Hematopoietic Progenitor Clonogenic Assay: Clonogenicity of mouse bone marrow cells was quantified in standard semisolid cultures using 1 mL of Methocult GF+ system for mouse cells (Stem Cell Technologies Inc., Vancouver, BC) according to the manufacturer's instructions. Briefly, colony forming units (CFU) were assayed on days 0 (2 h post-TBI), 1, 3, 7, 15, and 30 after 7 Gy exposure or unirradiated mice. Cells from three femurs from different animals were pooled, washed twice with IMDM and seeded at 1 to 5×10⁴ cells per 35 mm cell culture dishes (BD Biosciences, San Jose, Calif.). Each sample was plated in duplicate to be scored 14 days after plating. Granulocyte-macrophage colony forming units (CFU-GM), granulocyte-erythrocyte-monocyte-macrophage CFU (CFU-GEMM), colony-forming unit-erythroid (CFU-E) and erythroid burst-forming units (BFU-E) were identified and quantified following the manufacturer's instructions. Colonies were counted 14 days after plating using a Nikon TS100F microscope. Fifty or more cells were considered one colony. Data were expressed as mean f standard error of mean (SEM). Statistical significance was determined between irradiated vehicle treated and RWJ-800088-treated groups.

Sternal histopathology: Following blood collection, animals were euthanized, and the sterna were collected on 0 (2 h post-TBI), 1, 3, 7, 15 and 30 days post-TBI. The sterna were fixed in a 20:1 volume of fixative (10⁰/% buffered formalin) to tissue for at least 24 h and up to 7 days. Fixed sterna were decalcified for 3 h in 12-18% sodium EDTA (pH 7.4-7.5), and specimens were dehydrated using graded ethanol concentrations and embedded in paraffin. Longitudinal 5 μm sections were stained with regular hematoxylin and eosin (H&E) stain. A board-certified veterinary pathologist conducted blinded histopathological evaluation of these samples. The bone marrow was evaluated in situ within sternebrae and graded for total cellularity and megakaryocyte numbers averaged per 10 high power fields at 40× magnification using a BX41 Olympus microscope (Olympus Corporation, Minneapolis, Minn.). The grade scale used for cellularity is as follows: Grade 1: <10%; Grade 2: 11-30%; Grade 3: 31-60%; Grade 4: 61-89%; Grade 5: >90% (ref). Images were captured with an Olympus DP70 camera (Olympus Corporation, Minneapolis, Minn.) and imported into Adobe Photoshop (version CS5) for analysis.

Statistical analysis: Survival data was plotted as Kaplan-Meier plots. For the survival data, Fisher's exact test was used to compare survival at 30 days and a log-rank test was used to compare survival curves with GraphPad Prism 7 software. Means and standard errors were reported for all other data. Analysis of variance (ANOVA) was used to determine if there was a significant difference among different groups. A significance level was set at 5% for each test. IBM SPSS Statistics 22 software was used for probity analysis.

Results

Table 1 shows a summary of the survival results obtained in mice following TBI exposure with different doses and timing of administration of RWJ-800088. As seen in the survival differential column, there is a consistent survival benefit observed with RWJ-800088 compared to vehicle across multiple strains of mice, in both genders and with lethal and supra-lethal doses of radiation. More detailed results are presented in the sections that follow.

TABLE 1
Mouse Survival Data

| | | | | | | | | | | Platelet | | | |
|---|---|---|---|---|---|---|---|---|---|---|---|---|---|
| | | | | | | | | | | Elevation | | | |
| | | | | | | | | | TPOm | in Non- | | | |
| | | | | | | | | | Time of | Irradiated | | | |
| | | | | | | | | | First Dose | Animals | | | |
| | | | | | | | | TPOm | Relative | (fold | | | Survival |
| | Gender | | Radiation | TPOm | | TPOm | | Dose | to | increase | | Survival | Differential |
| | (M/F/ | | Dose | Dose | TPOm | # of | TPOm Dose | Duration | Irradiation | over | Survival | treat- | (Treatment- |
| Strain | Both) | n | (Gy) | (mg/kg) | Pre/Post | Doses | Frequency | (Days) | (hr) | baseline)^{a} | vehicle^{b} | ment | Vehicle) |
| C57BL/6 | M | 24 | 8.75 | 3 | Pre | 1 | Single Dose | 1 | −24 | 3.40 | 0% | 100% | 100% |
| C57BL/6 | F | 24 | 8.75 | 3 | Pre | 1 | Single Dose | 1 | −24 | 3.40 | 0% | 100% | 100% |
| C3H/HeN | M | 24 | 8.75 | 3 | Pre | 1 | Single Dose | 1 | −24 | 3.40 | 0% | 92% | 92% |
| CD2F1 | M | 24 | 9.25 | 0.3 | Pre | 1 | Single Dose | 1 | −24 | 3.90 | 42% | 83% | 41% |
| CD2F1 | M | 24 | 9.75 | 0.1 | Pre | 1 | Single Dose | 1 | −24 | 2.50 | 0% | 92% | 92% |
| CD2F1 | M | 24 | 9.75 | 0.3 | Pre | 1 | Single Dose | 1 | −24 | 3.90 | 0% | 92% | 92% |
| CD2F1 | M | 24 | 9.75 | 0.3 | Pre | 1 | Single Dose | 1 | −2 | 3.90 | 33% | 92% | 58% |
| CD2F1 | M | 24 | 9.75 | 0.3 | Pre | 1 | Single Dose | 1 | −12 | 3.90 | 4% | 100% | 96% |
| CD2F1 | M | 24 | 9.75 | 0.3 | Pre | 1 | Single Dose | 1 | −24 | 3.90 | 4% | 100% | 96% |
| CD2F1 | M | 24 | 9.75 | 1 | Pre | 1 | Single Dose | 1 | −24 | 4.30 | 0% | 92% | 92% |
| CD2F1 | M | 24 | 9.75 | 2 | Pre | 1 | Single Dose | 1 | −24 | 3.85 | 0% | 96% | 96% |
| CD2F1 | M | 24 | 9.75 | 3 | Pre | 1 | Single Dose | 1 | −24 | 3.40 | 0% | 100% | 100% |
| CD2F1 | M | 24 | 10.5 | 0.1 | Pre | 1 | Single Dose | 1 | −24 | 2.50 | 0% | 90% | 90% |
| CD2F1 | M | 24 | 10.5 | 0.3 | Pre | 1 | Single Dose | 1 | −24 | 3.90 | 0% | 95% | 95% |
| CD2F1 | M | 24 | 10.5 | 1 | Pre | 1 | Single Dose | 1 | −24 | 4.30 | 0% | 100% | 100% |
| CD2F1 | M | 24 | 10.5 | 3 | Pre | 1 | Single Dose | 1 | −24 | 3.40 | 0% | 90% | 90% |
| CD2F1 | M | 24 | 10.5 | 3 | Pre | I | Single Dose | 1 | −24 | 3.40 | 0% | 100% | 100% |
| CD2F1 | M | 24 | 11 | 0.1 | Pre | 1 | Single Dose | 1 | −24 | 2.50 | 0% | 54% | 54% |
| CD2F1 | M | 24 | 11 | 0.3 | Pre | 1 | Single Dose | 1 | −24 | 3.90 | 0% | 83% | 83% |
| CD2F1 | M | 24 | 11 | 0.3 | Pre | 1 | Single Dose | 1 | −24 | 3.90 | 0% | 83% | 83% |
| CD2F1 | M | 24 | 11 | 1 | Pre | 1 | Single Dose | 1 | −24 | 4.30 | 0% | 71% | 71% |
| CD2F1 | M | 24 | 11 | 1 | Pre | 1 | Single Dose | 1 | −24 | 4.30 | 0% | 96% | 96% |
| CD2F1 | M | 24 | 11 | 1 | Pre | 1 | Single Dose | 1 | −24 | 4.30 | 0% | 100% | 100% |
| CD2F1 | M | 24 | 11 | 1 | Pre | 1 | Single Dose | 1 | −12 | 4.30 | 0% | 100% | 100% |
| CD2F1 | M | 24 | 11 | 1 | Pre | 1 | Single Dose | 1 | −2 | 4.30 | 0% | 17% | 17% |
| CD2F1 | M | 24 | 11 | 3 | Pre | 1 | Single Dose | 1 | −24 | 3.40 | 0% | 100% | 100% |
| CD2F1 | M | 24 | 11 | 3 | Pre | 1 | Single Dose | 1 | −24 | 3.40 | 0% | 100% | 100% |
| CD2F1 | M | 24 | 11 | 3 | Pre | 1 | Single Dose | 1 | −24 | 3.40 | 0% | 100% | 100% |
| CD2F1 | M | 24 | 11.5 | 3 | Pre | 1 | Single Dose | 1 | −24 | 3.40 | 0% | 92% | 92% |
| CD2F1 | M | 24 | 11.75 | 3 | Pre | 1 | Single Dose | 1 | −24 | 3.40 | 0% | 75% | 75% |
| CD2F1 | M | 24 | 12 | 1 | Pre | 1 | Single Dose | 1 | −24 | 4.30 | 0% | 88% | 88% |
| CD2F1 | M | 24 | 12 | 3 | Pre | I | Single Dose | 1 | −24 | 3.40 | 0% | 83% | 83% |
| CD2F1 | M | 24 | 12.5 | 1 | Pre | 1 | Single Dose | 1 | −24 | 4.30 | 0% | 54% | 54% |
| CD2F1 | M | 24 | 12.5 | 3 | Pre | 1 | Single Dose | 1 | −24 | 3.40 | 0% | 46% | 46% |
| CD2F1 | M | 24 | 13 | 1 | Pre | 1 | Single Dose | 1 | −24 | 4.30 | 0% | 42% | 42% |
| CD2F1 | M | 24 | 13.5 | 1 | Pre | 1 | Single Dose | 1 | −24 | 4.30 | 0% | 13% | 13% |
| CD2F1 | M | 24 | 14 | 1 | Pre | 1 | Single Dose | 1 | −24 | 4.30 | 0% | 1% | 1% |
| CD2F1 | M | 24 | 9.25 | 0.3 | Post | 1 | Single Dose | 1 | 4 | 3.90 | 35% | 88% | 53% |
| CD2F1 | M | 24 | 9.3 | 0.3 | Post | 1 | Single Dose | 1 | 24 | 3.90 | 29% | 71% | 42% |
| CD2F1 | M | 24. | 9.3 | 1 | Post | 1 | Single Dose | 1 | 24 | 4.30 | 29% | 79% | 50% |
| CD2F1 | M | 24 | 9.35 | 0.1 | Post | 1 | Single Dose | 1 | 24 | 2.50 | 33% | 58% | 25% |
| CD2F1 | M | 24 | 9.35 | 0.3 | Post | 1 | Single Dose | 1 | 24 | 3.90 | 33% | 58% | 25% |
| CD2F1 | M | 24. | 9.35 | 0.3 | Post | 1 | Single Dose | 1 | 24 | 3.90 | 54% | 71% | 17% |
| CD2F1 | M | 24 | 9.35 | 1 | Post | 1 | Single Dose | 1 | 24 | 4.30 | 33% | 63% | 29% |
| CD2F1 | M | 24 | 9.35 | 2 | Post | 1 | Single Dose | 1 | 24 | 3.85 | 33% | 50% | 17% |
| CD2F1 | M | 24 | 9.35 | 3 | Post | 1 | Single Dose | 1 | 24 | 3.40 | 33% | 46% | 13% |
| CD2F1 | M | 24 | 9.75 | 0.1 | Post | 1 | Single Dose | 1 | 4 | 2.50 | 21% | 42% | 21% |
| CD2F1 | M | 24 | 9.75 | 0.3 | Post | 1 | Single Dose | 1 | 4 | 3.90 | 21% | 63% | 42% |
| CD2F1 | M | 24 | 9.75 | 1 | Post | 1 | Single Dose | 1 | 4 | 4.30 | 21% | 88% | 67% |
| CD2F1 | M | 24 | 9.75 | 1 | Post | 1 | Single Dose | 1 | 4 | 4.30 | 20% | 80% | 60% |
| CD2F1 | M | 24 | 9.75 | 1 | Post | 1 | Single Dose | 1 | 8 | 4.30 | 10% | 90% | 80% |
| CD2F1 | M | 24 | 9.75 | 1 | Post | 1 | Single Dose | 1 | 12 | 4.30 | 20% | 70% | 50% |
| CD2F1 | M | 24 | 9.75 | 3 | Post | 1 | Single Dose | 1 | 4 | 3.40 | 21% | 54% | 33% |
| CD2F1 | M | 24 | 9.35 | 0.3 | Post | 2 | 12 hours apart | 1 | 24 | 3.90 | 33% | 54% | 21% |
| CD2F1 | M | 24 | 9.35 | 0.3 | Post | 3 | 8 hours apart | 1 | 24 | 3.90 | 42% | 50% | 8% |
| C57BL/6 | Both | 20 | 7.76 | 0.3 | Post | 1 | Single Dose | 1 | 6 | 3.90 | 75% | 100% | 25% |
| C57BL/6 | Both | 20 | 7.76 | 0.3 | Post | 1 | Single Dose | 1 | 24 | 3.90 | 75% | 90% | 15% |
| C57BL/6 | Both | 20 | 7.96 | 0.3 | Post | 1 | Single Dose | 1 | 6 | 3.90 | 55% | 100% | 45% |
| C57BL/6 | Both | 20 | 7.96 | 0.3 | Post | 1 | Single Dose | 1 | 24 | 3.90 | 55% | 75% | 20% |

^{a}Platelet elevation in non-irradiated animals for TPOm doses of 2 mg/kg are imputed as the mean platelet elevation observed in the 1 mg/kg and 3 mg/kg cohorts.
^{b}Survival following vehicle is assumed to be 0% in treatments of at least 10.5 Gy when no control cohort was performed within study under the same conditions.

Effects of Prophylactic RWJ-800088 Administration 24 h Pre-TBI in CD2F1 Male Mice

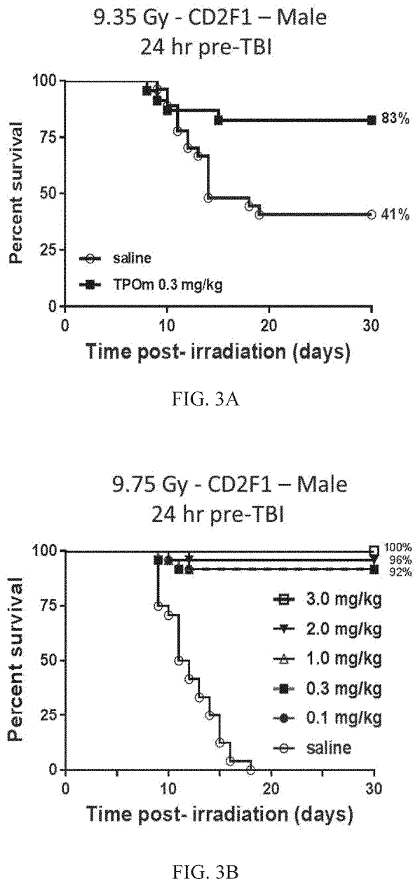

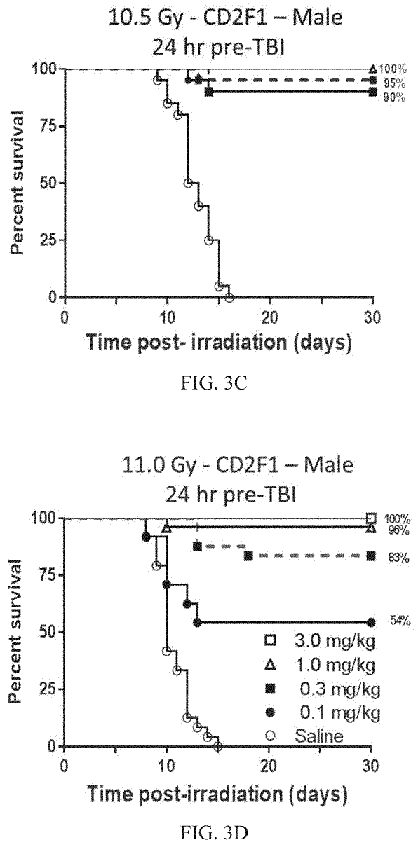

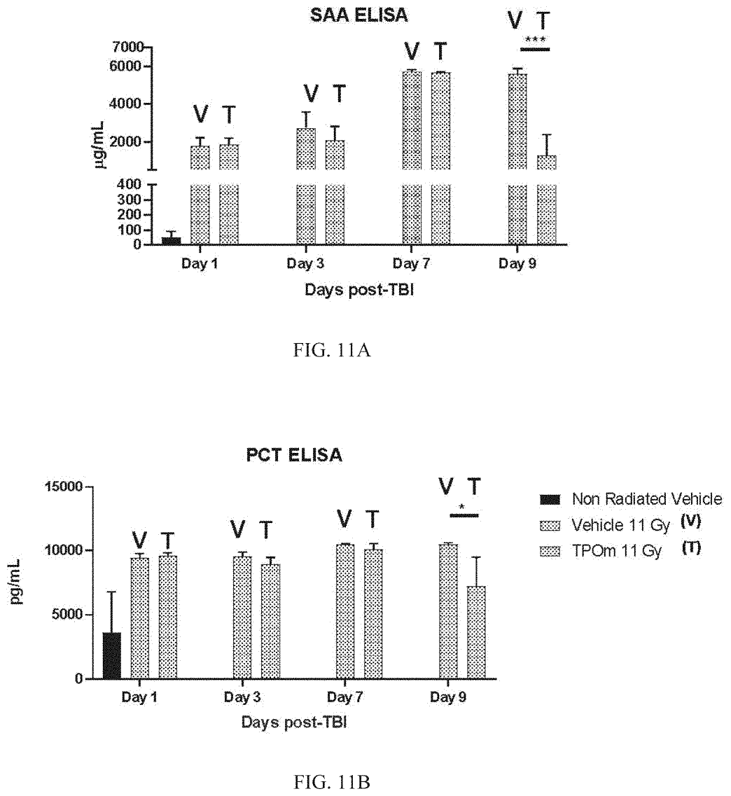

To investigate the effects of RWJ-800088 administered 24 h prior to total body irradiation (pre-TBI), CD2F1 male mice (24 mice/group) were treated with 0.3 mg/kg or 0.1-3 mg/kg of RWJ-800088 and then irradiated with 9.35 Gy (˜LD70/30 dose) (FIG. 3A), 9.75 Gy (FIG. 3B), 10.5 Gy (FIG. 3C) and 11 Gy (FIG. 3D) at an estimated dose rate of 0.6 Gy/min. Following a dose of 9.35 Gy there was 42% survival of the saline treated group compared to 83% survival in the RWJ-800088 (0.3 mg/kg) treated group (Log-rank test p=0.0061). At 9.75 Gy all of the mice from the saline group died by 18 hours post-TBI and 92-100% survival was observed for the RWJ-800088 treated groups (FIG. 3B). There was no dose dependent separation. At 10.5 Gy, no significant difference in percent survival (90-100%) among the range of RWJ-800088 doses (0.1-3 mg/kg), whereas all the mice in saline group died by day 16 post-TBI (FIG. 3C). Total body irradiation of 11.0 Gy resulted in 54%, 83%, 96% and 100% survival at 0.1, 0.3, 1 and 3 mg/kg RWJ-800088 respectively, whereas all mice from the saline (vehicle) group died by day 15 post-TBI (FIG. 3D). The highest survival efficacy was found with 3 mg/kg dose of RWJ-800088 with no statistically significant difference with 1 mg/kg.

Accelerated Recovery from Radiation-Induced Pancytopenia with RWJ-800088 in CD2F1 Mice

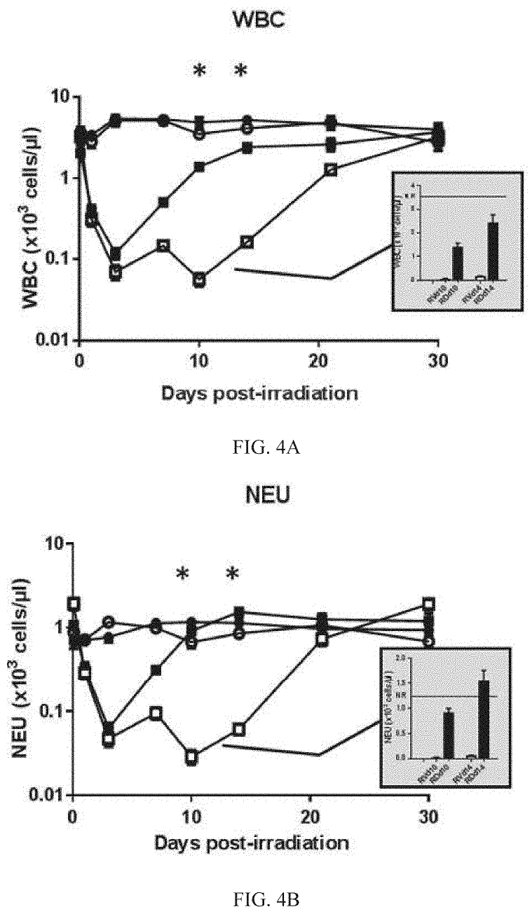

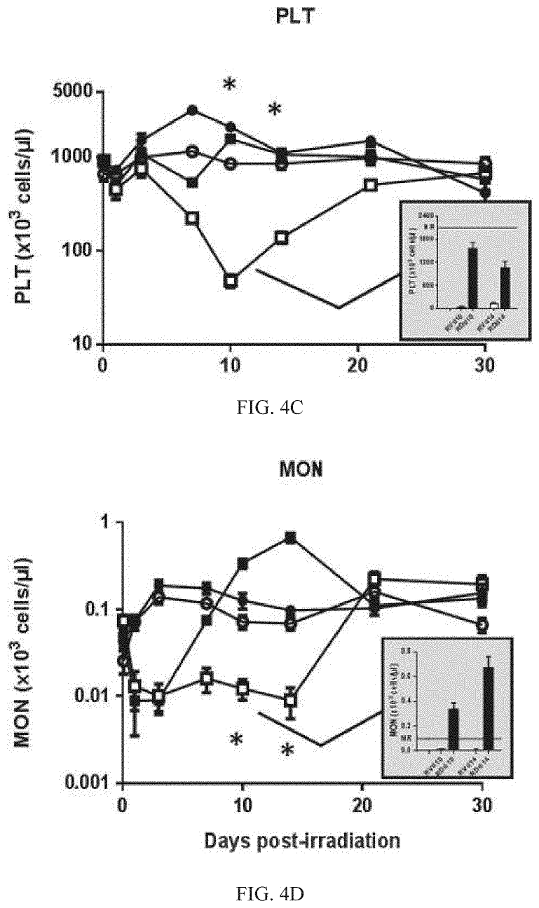

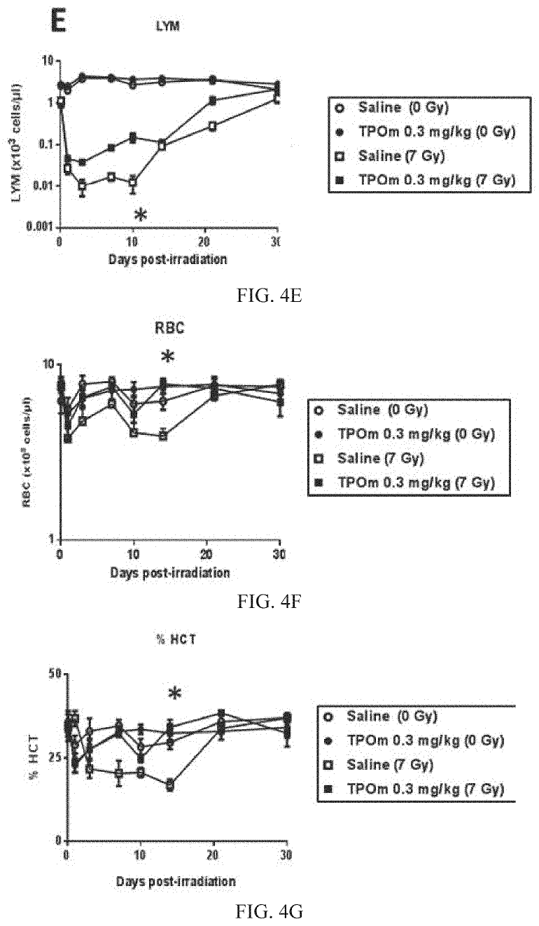

Peripheral blood cell recovery was studied by measuring blood cell counts, white blood cells (WBC), red blood cells (RBC), % Hematocrit (% HCT), neutrophils, platelets (PLT), monocytes (MON) and lymphocytes (LYM) of non-irradiated groups and comparing them to that of irradiated groups either treated with saline (vehicle control) or RWJ-800088 (FIGS. 4A-G). In the irradiated groups, by day 3 post-TBI, decline in blood cell counts were observed in both saline and RWJ-800088 treated groups. The recovery from cytopenia of all blood cell counts were significant in the RWJ-800088 treated group, when compared to the vehicle control group. In the non-irradiated groups, significant (p<0.05) increase in PLT was observed in RWJ-800088 treated groups compared to saline on days 7 and 10 post-TBI (days 8 and 11 after RWJ-800088 administration.

The effects on the peripheral blood cell counts and circulating Erythropoietin and FLT3 ligand were as follows:

White Blood Cells: The white blood cell (WBC) counts decreased sharply reaching a nadir (0.056×10³±0.009×10³ cells/μL) in the irradiated saline control group on day 10 post-TBI. On days 7, 10 and 14 post-TBI (FIG. 4A), the RWJ-800088 treated group showed significant recovery (day 7: 0.5±0.037 cells/μL; day 10: 1.39±0.1756 cells/μL; and day 14: 2.43±0.32 cells/μL) as compared to the vehicle treated group (day 7: 0.15×10³+0.0189×10³ cells/μL; day 10: 0.056×10³±0.0097×10³ cells/μL; and day 14: 0.16×10³±0.0174×10³ cells/μL). WBC counts in the irradiated vehicle-treated group remained low until day 10 post-TBI, with a slow recovery profile; whereas the cells in the RWJ-800088 treated group recovered faster. By day 30, all four groups had similar WBC cell counts, as the irradiation dose was non-lethal.

Neutrophils: Neutrophil (NEU) counts decreased sharply reaching a neutropenia nadir (0.029×10³±0.0051×10³ cells/μL) in the irradiated saline control group on day 10 post-TBI. On days 7, 10 and 14 post-TBI (FIG. 4B), the RWJ-800088 treated group showed significant recovery from neutropenia (day 7: 0.31±0.026 cells/μL; day 10: 0.92 t 0.0721 cells/μL; and day 14: 1.54±0.21 cells/μL) as compared to the vehicle treated group (day 7: 0.095×10³±0.0147×10³ cells/μL; day 10: 0.029×10³±0.0051×10³ cells/μL; and day 14: 0.06×10³±0.0079×10³ cells/μL). NEU counts in the irradiated vehicle-treated group remained low until day 10 post-TBI, with a slow recovery profile; whereas the cells in the RWJ-800088 treated group recovered by Day 10. By day 30, all four groups had similar NEU cell counts with complete recovery.

Platelets: The platelet (PLT) nadir was reached for the irradiated vehicle-treated group on day 10 (48×10³+7.12×10³ cells/μL), but the RWJ-800088 treated group had a significantly higher (p<0.0001) cell count (1565×10³±148×10³ cells/μL) (FIG. 4C), protecting the mice from thrombocytopenia. By day 14, there was no difference between the non-irradiated RWJ-800088 and the irradiated RWJ-800088 treated group (1070×10³±156×10³ cells/μL) (FIG. 4C). PLT cell numbers in non-irradiated groups were found to be significantly different based on the treatment they received (either saline or RWJ-800088). Significantly higher PLT induction after RWJ-800088 treatment in the control group could be one of the possible mechanisms by which RWJ-800088 helps peripheral blood cell recovery and supports faster recovery from radiation induced thrombocytopenia leading to survival of the animals.

Monocyte and Lymphocyte: The irradiated group receiving RWJ-800088 showed markedly higher monocyte (MON) counts than the irradiated vehicle-treated group on days 7, 10 and 14 post-TBI, and the differences were statistically significant (p<0.001) (FIG. 4D). These results indicated that the administration of RWJ-800088 improved peripheral blood monocyte counts in irradiated mice. There were significant (p<0.05) increases in lymphocyte (LYM) as well when mice were treated with RWJ-800088 compared to the vehicle-treated group on day 10 post-TBI (FIG. 4E).

Red Blood Cells and Percent Hematocrit: Changes in the red blood cell (RBC) count and percent hematocrit (% HCT) in the different groups are shown in FIGS. 4F and 4G. On day 14 the % HCT of the irradiated vehicle-treated group was significantly lower than that of the control group or irradiated RWJ-800088 treated group (p<0.001). The same effect was also observed in the RBC counts, suggesting recovery of peripheral hematopoietic cells with RWJ-800088 treatment in irradiated mice.

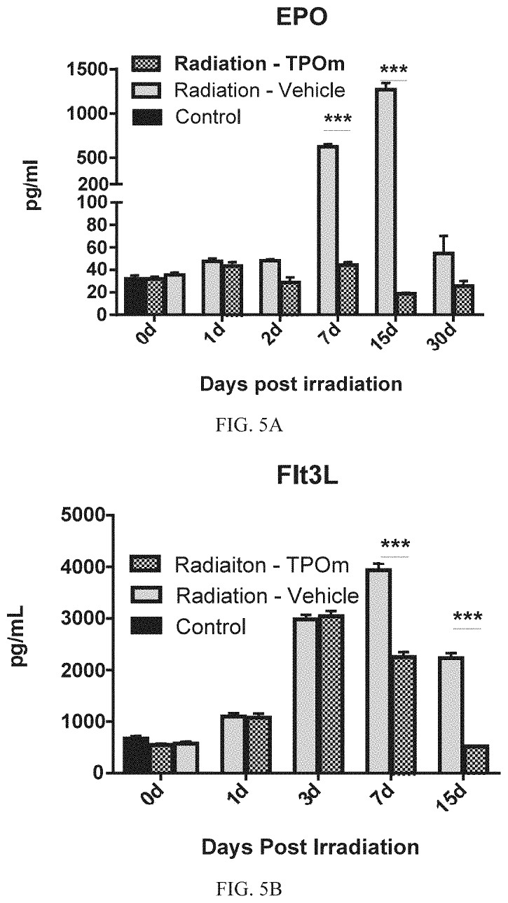

Erythropoietin and FLT3 Ligand: Consistent with the greater nadir and more rapid recovery of the RBCs and white blood cells (WBCs), the circulating levels of erythropoietin (EPO) (FIG. 5A) and FLT3 ligand (FIG. 5B) were significantly (p<0.0001) lower in the RWJ-800088 treated irradiated mice compared to the vehicle treated irradiated mice. The concentration of erythropoietin remained consistent with the non-irradiated control animals, and while the concentration of FLT3 ligand was elevated in the RWJ-800088 group and similar to the irradiated vehicle treated animals, it was substantially lower on Day 7 and returned to pre-treatment levels by Day 15 while the levels remained significantly elevated in the irradiated vehicle treated mice. The effects of RWJ-800088 on accelerating the recovery of the hematopoietic system is evident in the modulation of these cytokine biomarkers of normal hematopoiesis.

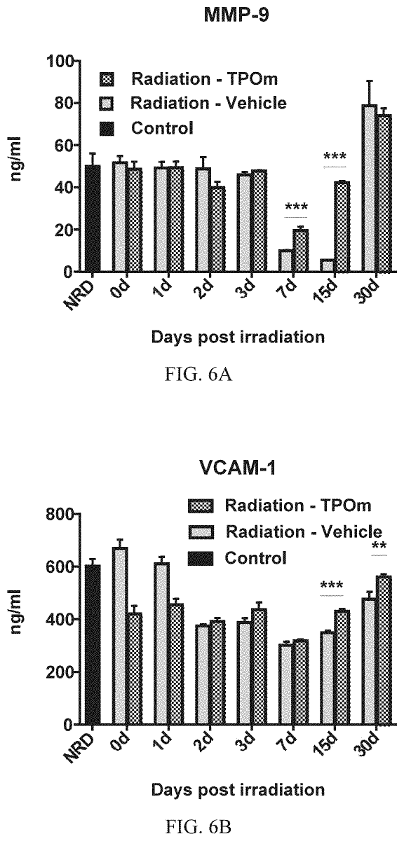

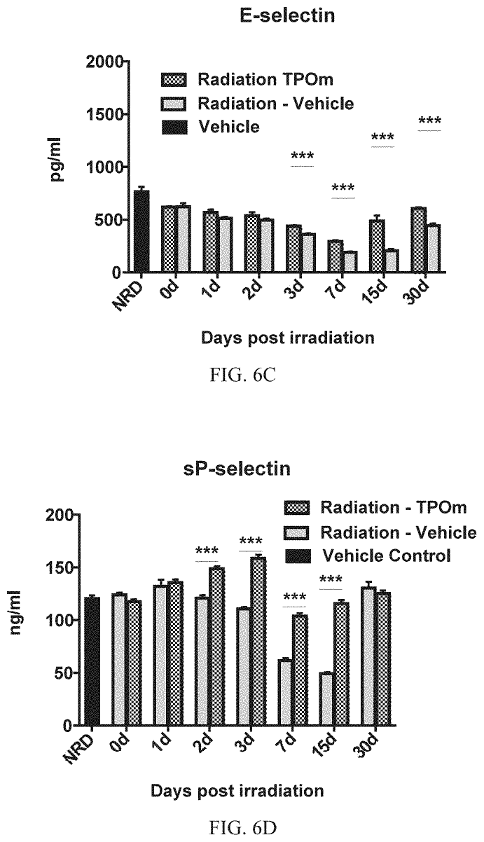

MMP9, VCAM-1, E-Selectin, P-Selectin: There were statistically significant increases (p<0.0001) in the RWJ-800088 treated mice compared to vehicle with respect to circulating levels of MMP-9 on days 7 and 15 (FIG. 6A), VCAM-1 on days 15 and 30 (FIG. 6B), E-Selectin (FIG. 6C) on days 3, 7 15 and 30 and sP-Selectin on days 2, 3, 7, and 15 (FIG. 6D) post-irradiation.

Effects of RWJ-800088 on Hematopoietic Progenitor Cells when Administered 24 h pre-TBI

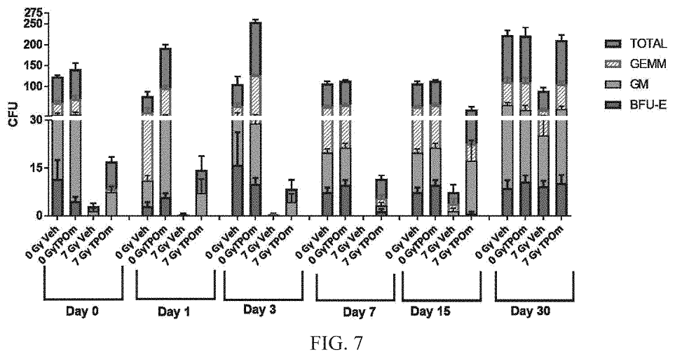

In addition to detrimental effects on peripheral blood cells, irradiation also negatively affects the hematopoietic progenitor cells in bone marrow. Clonogenic assays were carried out to evaluate the extent of damage caused by irradiation and possible recovery by RWJ-800088 treatment administered 24 h pre-TBI. Colony forming unit (CFU) assays measured CFU-GM, CFU-GEMM, CFU-E and BFU-E to evaluate the function of hematopoietic cells. Prior to day 15 post-TBI at 7 Gy, no colonies were observed in the irradiated saline treated group (FIG. 7). On day 15, the total number of colonies found in the vehicle-treated group was significantly lower compared to the RWJ-800088 treated group. Even on day 30, the difference between the vehicle-treated and RWJ-800088 treated groups with respect to GM, GEMM, BFU-E and CFU-E was significantly lower (p<0.0001) (FIG. 7). This data suggests that the bone marrow cellular functions affected by irradiation in hematopoietic progenitor cells can be restored by RWJ-800088 treatment.

Effects of RWJ-800088 on Bone-Marrow Cellularity when Administered 24 h Pre-TBI

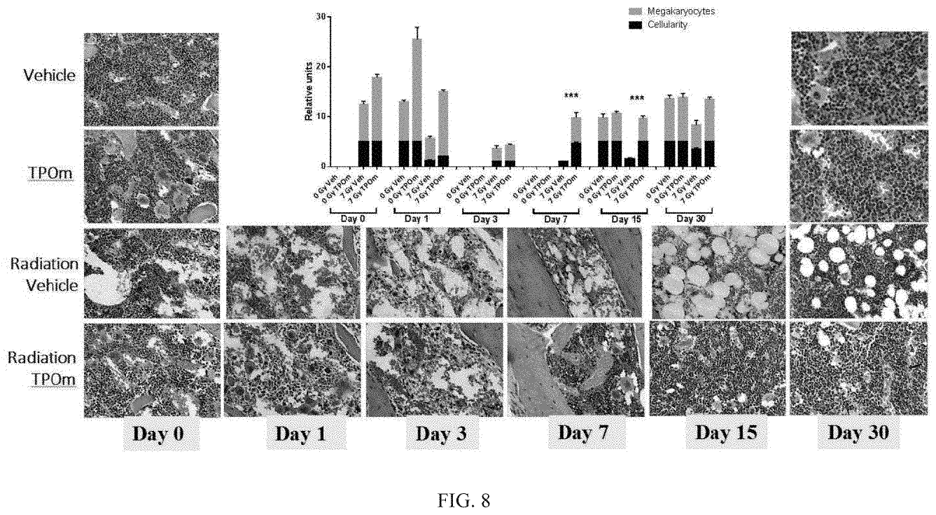

Bone marrow cellularity and architecture of CD2F1 mice treated 24 h pre-TBI with vehicle or RWJ-800088 were evaluated by the AFRRI pathologist (FIG. 8). Bone marrow cellularity was determined by evaluating the amount of adipose (fat) tissue versus hematopoietic cells (minus, mature red blood cells) on one (10×) high power field (HPF). In order to score cellularity, a grade was assigned, which correlated with a “percentage range” of cellularity; an average was obtained for each group. The grading scheme was: Grade 1: <10%; Grade 2: 11-30%; Grade 3: 31-60%; Grade 4: 61-89%; Grade 5: >90% cellularity (FIG. 8). Irradiated saline treated group (irradiated vehicle-treated—RV, irradiated RWJ-800088 treated—RD) was compared to the respective non-irradiated controls (non-irradiated vehicle-treated NRV, non-irradiated RWJ-800088 treated NRD).

Samples were collected on different days post-TBI up to day 30. The extent of recovery from radiation damage was estimated from the H&E stained slides and quantitated as number of megakaryocytes and percent cellularity (FIG. 8). Megakaryocytes were evaluated by averaging the number of cells per 10 (40×) high power fields (HPFs). When compared to non-irradiated controls (NRV or NRD), irradiated samples show significant damage in the vehicle treated group in comparison to the RWJ-800088 treated group on day 1. On Day 1, megakaryocytes counts were significantly lower in the both irradiated groups. However, by day 7, the RWJ-800088 treated group showed significant recovery. By day 15, there were significant differences in the irradiated vehicle-treated and drug treated groups with respect to number of megakaryocytes as well as % cellularity. By day 30, even though the vehicle treated groups recovered, cellularity remained lower than the drug treated group. This demonstrates the accelerated recovery of the hematopoietic system in response to treatment with RWJ-800088.

Effects of RWJ-800088 on Survival and Recovery from Gastrointestinal Injury at Supra-Lethal Doses of TBI to CD2F1 Mice

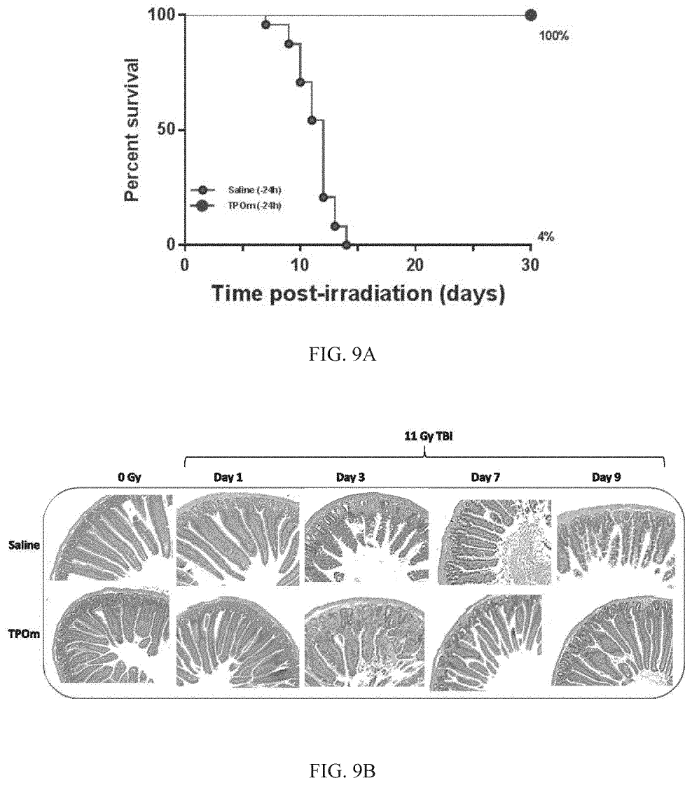

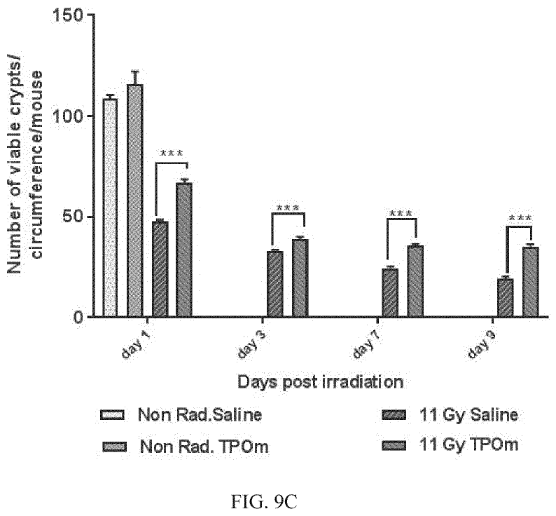

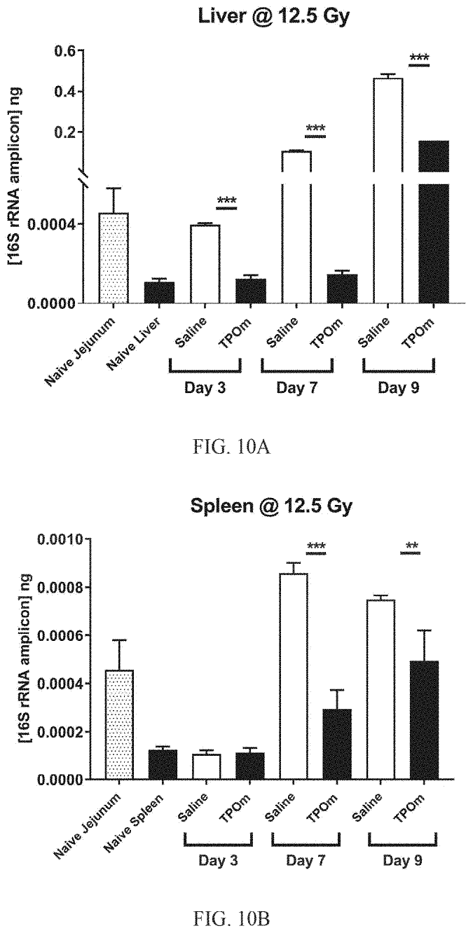

CD2F1 male mice (8 mice/group/time-point) exposed to TBI (11 Gy), received either RWJ-800088 (1 mg/kg) or saline 24 h prior to TBI. Jejunum samples were collected at days 1, 3, 7, 9 post-TBI. Representative sections were stained with H&E. There was a 100% increased survival in the RWJ-800088 treated mice compared to the saline treated animals (FIG. 9A—Kaplan Meier plot). There was also a significant increase in the number of viable crypts (FIG. 9C) and integrity of the jejunum (FIG. 9B) based on histological examination. RWJ-800088 also significantly reduced bacterial translocation to the liver (FIG. 10A) and spleen (FIG. 10B) following TBI when administered 24 hours prior to TBI. Further supporting a protective effect on the gut, there was a significant reduction in circulating sepsis biomarkers, serum amyloid A (FIG. 11A) and procalcitonin (FIG. 11B) nine days post-TBI when RWJ-800088 was administered 24 hours prior to TBI compared to vehicle.

Survival Increase with RWJ-800088 Administration Pre-TBI in C57Bl/6 Male and Female and C3H/HeN Male Mice

The survival efficacy of RWJ-800088 was tested in C57BL/6 (another strain of mice with different radiation sensitivity compared to CD2F1) in males and females. RWJ-800088 was administered (3 mg/kg) to C57BL/6 male (n=24) and female (n=24) mice 24 h prior to irradiation at 8.75 Gy (LD100/30). All animals in saline treated groups (males and females) died whereas there was no mortality in RWJ-800088 treated groups 30 days post-TBI. The survival efficacy of RWJ-800088 (3 mg/kg) was also observed in C3H/HeN male (n=24) mice irradiated at 8.75 Gy (LD100/30) (2). All animals in saline treated groups died whereas there was 92% survival observed in the RWJ-800088 (24 h pre-TBI) treated group 30 days post-TBI.

Effects of RWJ-800088 (1 mg/kg) Administration from 24 Hours Pre-TBI to 24 Hours Post-TBI in CD2F1 Male Mice

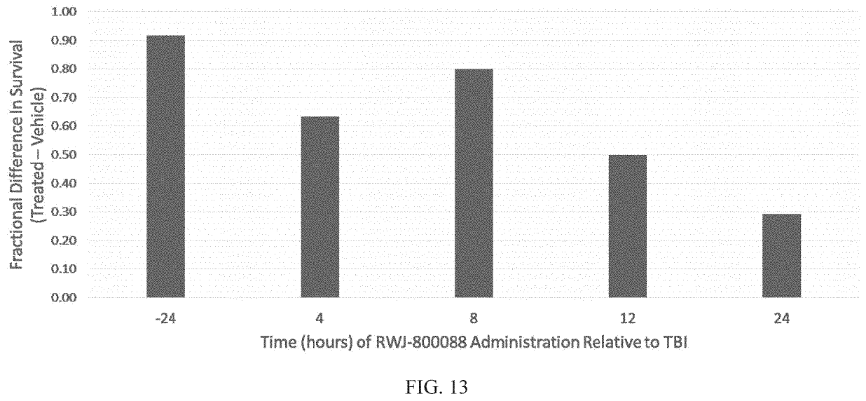

The survival difference compared to vehicle of RWJ-800088 (1 mg/kg) when administered from 24 hours pre-TBI to 24 hours post TBI was greatest with pre-TBI treatment and lowest 24-hours post treatment and generally decreased across the time range with the exception of the 8-hour time point (FIG. 13). The enhancement in survival efficacy of RWJ-800088 was almost 100% when administered 24 h prior to radiation exposure.

Dose Dependence of Survival Increase with RWJ-800088 when Administered Post-TBI in CD2F1 Mice

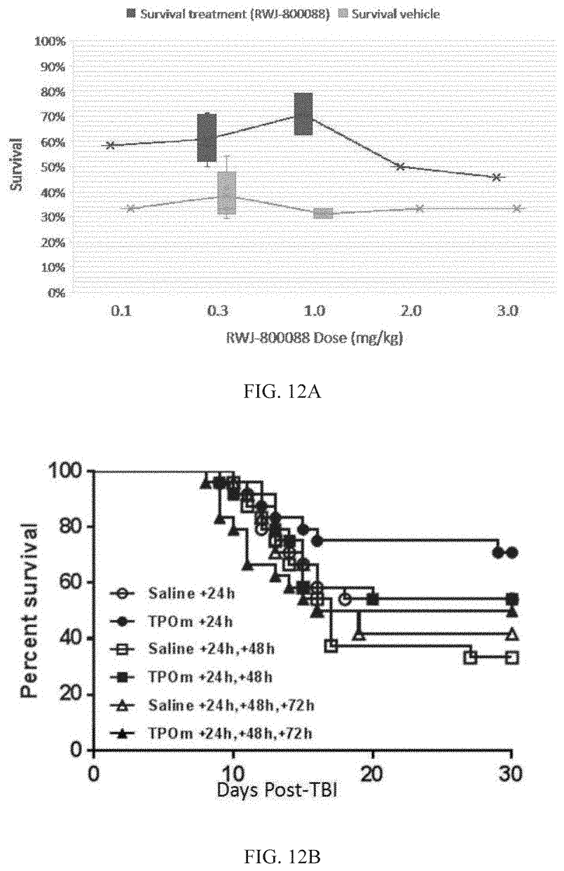

When RWJ-800088 was administered 24 hours post-TBI (9.3 Gy (˜LD70/30)) to CD2F1 mice, at doses ranging from 0.1 to 3 mg/kg there was an increase in survival from 0.1 to 1 mg/kg and then a slight reduction in the survival benefit at 2 and 3 mg/kg (FIG. 12A).

To confirm the survival benefit provided by RWJ-800088 administration following TBI in another mouse strain with difference in radiation sensitivity, C57BL/6 male mice were irradiated at 8.0 Gy (˜LD70/30) followed by administration of a single subcutaneous dose of RWJ-800088 (1 mg/kg) at 24 h after TBI. The percentage of mice surviving on day 30 post-TBI for the RWJ-800088 treated group was 83% and for the saline treated group it was only 13% (2).

The survival benefit shown from CD2F1 and C57BL/6 mice when compared to the respective vehicle-treated groups is statistically significant with log-rank test p values ranging from <0.0001-0.005. These results indicate that RWJ-800088 acts as an effective mitigator against radiation induced morbidity and mortality in two different strains of mice with differential radiation sensitivity (male CD2F1 and C57BL/6) and that the range of 0.3-1.0 mg/kg RWJ-800088 as the optimum single dose for RWJ-800088 as mitigator of radiation induced mortality in mice.

Survival Following Single Vs. Multiple Doses of RWJ-800088 in CD2F1 Mice Post-TBI

To investigate the effects of multiple doses of RWJ-800088 administered post-TBI, CD2F1 mice (24 males/group) were irradiated with 9.35 Gy (LD70/30 dose) and treated with 0.3 mg/kg/dose RWJ-800088 SC at either 24 h, 24 h+48 h, or 24 h+48 h+72 h post-TBI. There were 24 animals per treatment group for RWJ-800088 and the vehicle. The mice were monitored daily for 30 days and euthanized in moribund condition according to the predetermine health score previously described. The highest percent survival was with 1-dose regimen of RWJ-800088 at 24 h post TBI (71%) as compared to its saline control (54%), however, due to the higher rate of survival in the saline group following a LD70 radiation dose, was not statistically significant (FIG. 12A). In the case of 2- and 3-dose regimens, the survival benefit due to RWJ-800088 injection (54% and 50% respectively) was not significantly higher than the respective saline controls (33% and 42% respectively) (FIG. 12B).

Dose Reduction Factor when RWJ-800088 (1 mg/kg) is Administered 24 Hours Post TBI and 24 Hours Pre-TBI to CD2F1 Male Mice

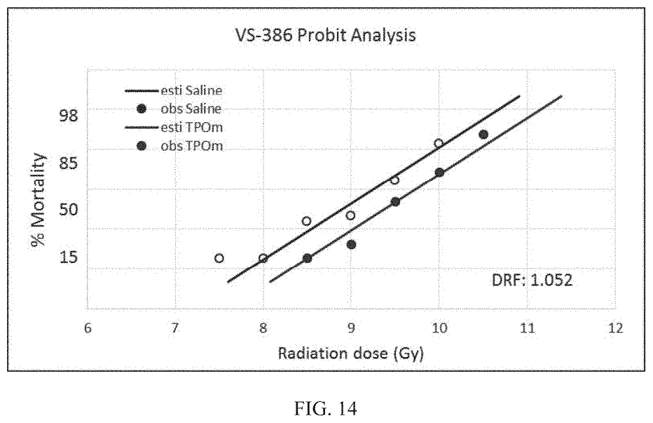

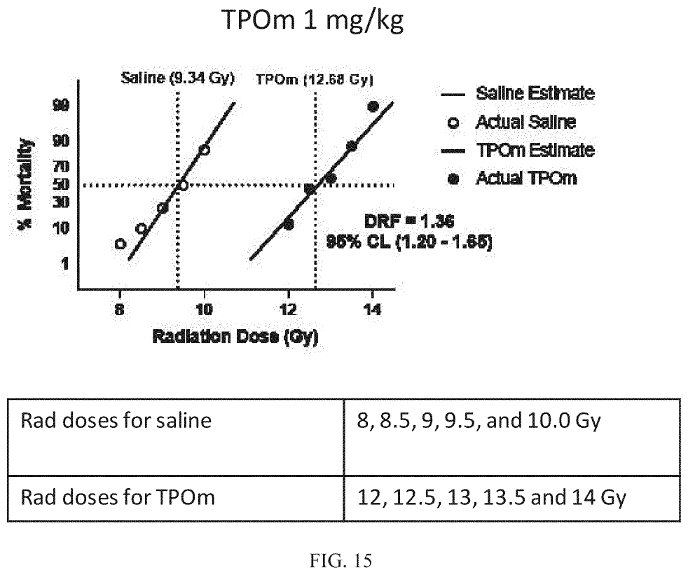

The Dose Reduction Factor when RWJ-800088 was administered 24 h pre- (FIG. 15) and post-TBI (FIG. 14) to male CD2F1 is 1.38 and 1.05, respectively. These data demonstrate an enhanced survival benefit at both time points but administering 24 h prior to TBI provides a greater benefit as compared to 24 h post-treatment time point.

### Example 3: Translation of the Dose that Produces Enhanced Survival and/or Organ and/or Vascular Protection from Animals to Humans with RWJ-800088

Rat Whole Body Irradiation Study

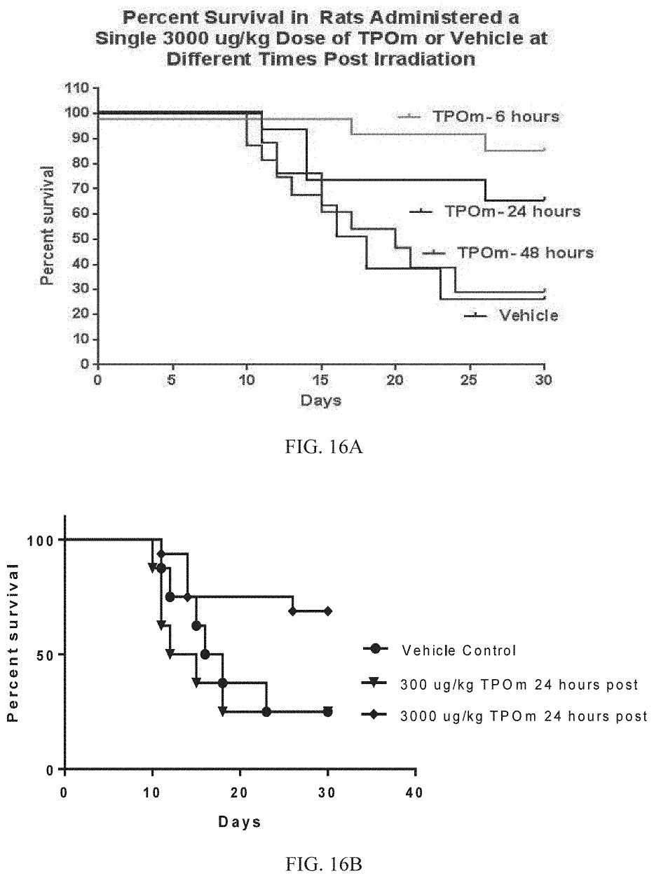

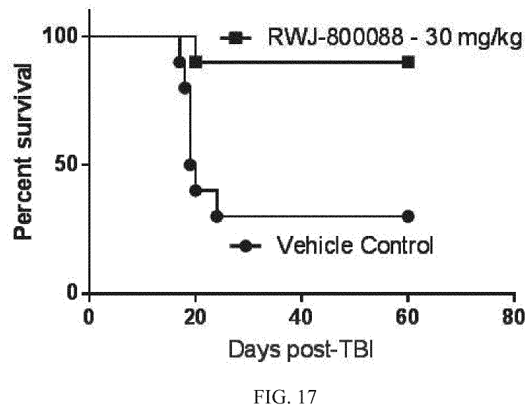

RWJ-800088 was administered at a 3000 μg/kg dose by SC injection to female rats (n=8) at 6, 24, or 48 hours and additionally 300 μg/kg dose at 24 hours after exposure to an LD70 total body dose of gamma radiation (Gammacell 3000 irradiator). The survival of animals administered RWJ-800088 at 3000 μg/kg at 6 and 24 hours was similar and was significantly higher than animals administered vehicle and RWJ-800088 at 48 hours post-irradiation (FIG. 16A). In addition, survival was substantially increased in the animals that received 3000 μg/kg compared with animals that received 300 μg/kg RWJ-800088 administered 24 hours post irradiation (FIG. 16B).

Dog Pilot Whole Body Irradiation Study

A pilot radio-mitigation study was conducted in dogs with RWJ-800088. These are initial results as dosage has not been optimized for dogs and they have been shown to be less sensitive to the platelet elevating effects of RWJ-800088 compared with other species. Results presented in Table 3 suggest that dogs in the RWJ-800088 group generally performed better than those in the vehicle control group.

TABLE 3
Effects Following a Single 10 mg/kg Dose of
RWJ-800088 (RWJ-800088) or Vehicle
Administered to Dogs 24 Hours after Whole
Body Radiation at an LD50 Exposure

| | | RWJ-800088 |
|---|---|---|
| Endpoint | Vehicle | Treated |
| Mortality | 9/12 | 7/12 |
| Mean survival time (days) | 17 | 36 |
| Diarrhea | 4/12 | 2/12 |
| Blood transfusions | 2.25/ | 1.25/ |
| | animal | animal |
| Severe neutropenia duration | 8.3 | 5.4 |
| Neutropenia nadir | 0.02 × 10⁹/L | 0.09 × 10⁹/L |
| Days of febrile neutropenia | 2.92 | 1.08 |
| Mild to severe hematopoietic bone | 10/12 | 6/12 |
| marrow hypocellularity | | |
| Mild to moderate necrohemorrhagic | 10/12 | 6/12 |
| bronchioalveolar or minimal subacute | | |
| bronchioalveolar inflammation | | |

Non-Human Primate (NHP) Whole Body Irradiation Studies

A pilot PK/PD study was performed to evaluate the efficacy of RWJ-800088 in rhesus monkeys (Study No. 2016-2693—CiToxLAB North America). Rhesus monkeys (n=10/group, 5 male/5 female) were treated with vehicle or RWJ-800088 (a single dose of 30 mg/kg RWJ-800088) administered 24 hours post TBI gamma radiation (600 cGY). FIG. 17 shows that RWJ-800088 had enhanced survival of 9/10 surviving vs. 3/10 in the vehicle. The ratio of the maximum platelet count to baseline following escalating doses of RWJ-800088 to healthy rhesus monkeys is as follows: 0.97±0.06× at 0.5 mg/kg, 1.08±0.15× at 2 mg/kg, 1.98±0.16× at 10 mg/kg, 2.9±0.03× at 20 mg/kg, and 3.83±0.11× at 40 mg/kg). These results support a dose dependent increase in platelet counts that reaches 2.5-4× baseline between 20 and 40 mg/kg. The 30 mg/kg dose was selected for the survival data shown in FIG. 17.

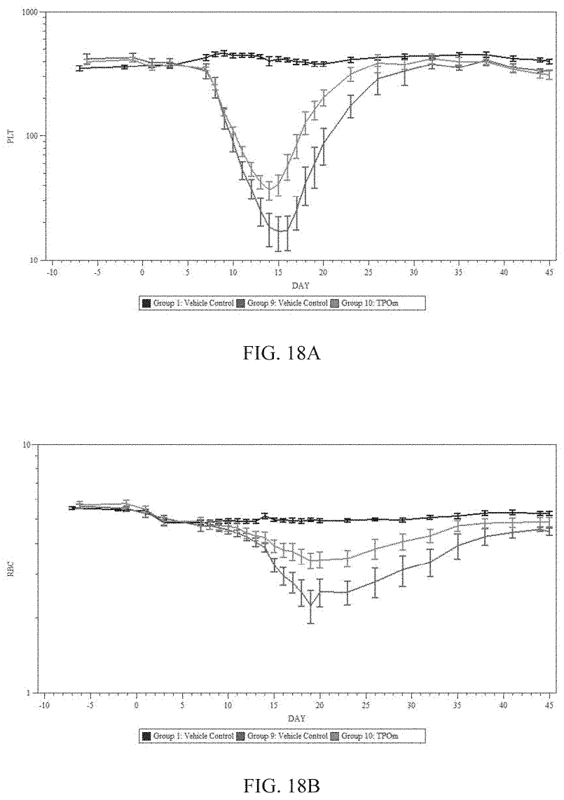

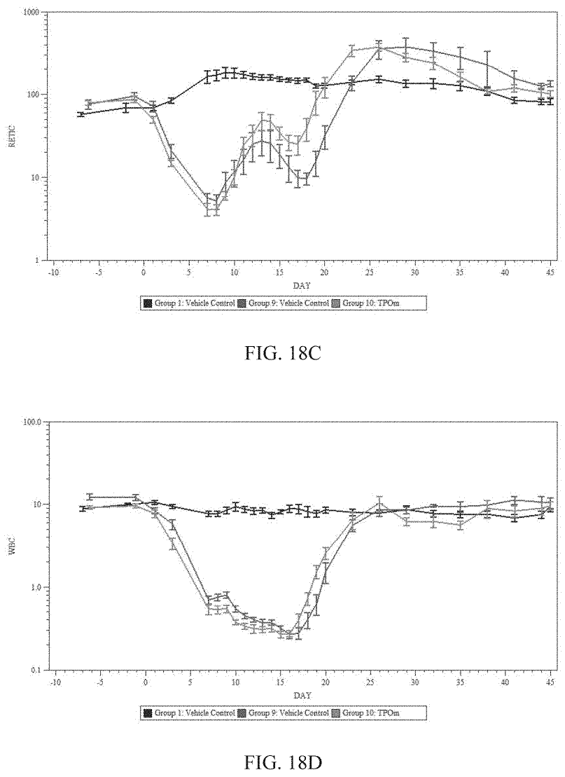

RWJ-800088 had beneficial effects on platelet counts in sham or irradiated animals with respect to nadir and recovery (FIG. 18A); irradiated animals treated with RWJ-800088 had a less severe decrease in RBC (FIG. 18B), reticulocytes (FIG. 18C) and WBC (FIG. 18D) compared to irradiated animals treated with vehicle. There is evidence of an increase in the nadir and recovery with RWJ-800088. These data with RWJ-800088 are consistent with published data showing that human recombinant thrombopoietin (rhTPO) treatment significantly promoted hematopoietic recovery and improved quality of life. Since this study was intended to be a PK/PD study and was not blinded and not powered to assess mortality, it is not determined if the enhanced survival across treatment groups is significant.

Clinical Study Results

RWJ-800088 has been investigated in two Phase 1 human studies. A First in Human (FIH) Phase 1 study (NAP1001) was conducted in healthy men and a Phase 1b study (NAP1002) was conducted in cancer patients being treated with platinum-based chemotherapy.

Study NAP1001: Single Dose Study in Healthy Men

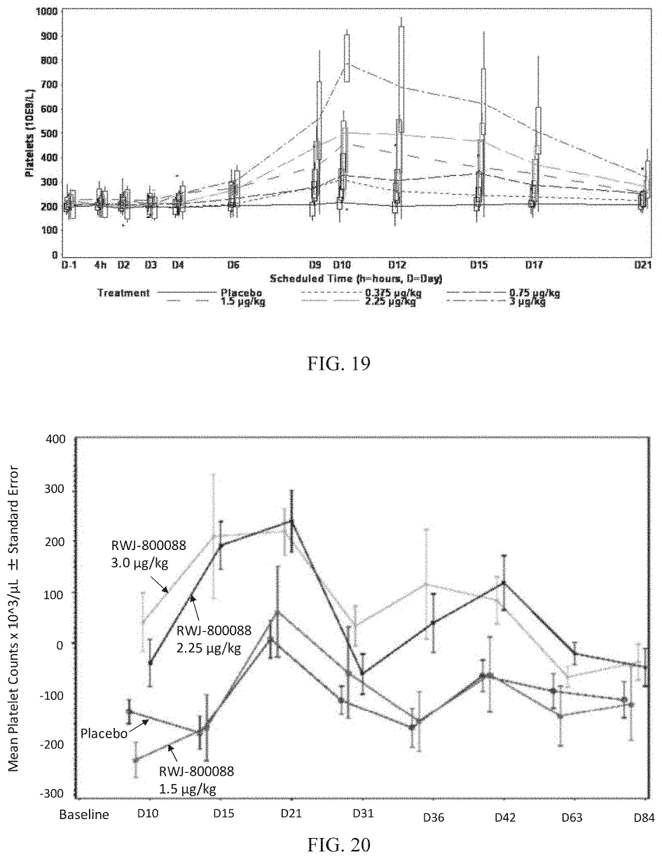

In the FIH, single dose, Phase 1 clinical study NAP1001, 40 healthy men were enrolled, and 30 subjects received a single IV dose of RWJ-800088 as a 5-mg/mL solution in saline (dose range 0.375 to 3 μg/kg); 10 subjects received placebo. Single IV doses of RWJ-800088 up to and including 3 μg/kg were well tolerated in healthy men with no apparent drug-related effects on adverse events, or cardiovascular or laboratory safety parameters (excluding platelet counts). Thirty-three (83%) of 40 subjects reported at least 1 treatment-emergent adverse event throughout the study. Similar proportions of subjects reported treatment-emergent adverse events following administration of placebo (9 [90%] of 10 subjects) and RWJ-800088 (24 [80%] of 30 subjects). The incidence of adverse events was not dose related. Mean platelet counts increased with increasing RWJ-800088 dose compared with placebo from Day 6 onwards, peaking at Days 10 to 12 before gradually returning to baseline by Day 21 (FIG. 19).

Study NAP1002: Multiple-Dose Study in Cancer Patients

In the second randomized, double-blind Phase 1 study (NAP1002), 46 subjects with cancer receiving platinum-based chemotherapy were enrolled into 3 cohorts: 12 subjects received 1.5 μg/kg RWJ-800088, 12 subjects received 2.25 μg/kg, 10 subjects received 3 μg/kg, and 12 subjects received placebo within 2 hours before administration of platinum-based chemotherapy on Day 1 of the first of 2 chemotherapy cycles. There was a 21-day interval between each chemotherapy cycle.

Treatment with RWJ-800088 (1.5, 2.25, and 3 μg/kg) was well-tolerated with a safety profile like that expected from concurrent treatment with platinum-based chemotherapy. There were no apparent drug-related adverse events (except for 1 serious adverse event of thrombocythemia), vital signs, or clinical laboratory parameters (excluding platelet counts).

Platelet results for the 3 dose groups are presented in Table 2 and Table 3 and shown in FIG. 20. There is clear evidence of protection against a drop in platelet counts at the doses of 2.25 and 3.0 μg/kg. Nadir and peak mean platelet counts were similar in subjects who received placebo or 1.5 μg/kg of RWJ-800088. However, subjects who received 2.25 or 3.0 μg/kg of RWJ-800088 had nadir and peak platelet counts that were approximately 2-fold higher than those of subjects who received placebo.

TABLE 2
Minimum Platelet Counts in Subjects with Cancer
Receiving Platinum-Based Chemotherapy
(Study NAP1002: All Randomized Subjects Analysis Set)

| | | | Geometric | GMR | 95% |
|---|---|---|---|---|---|
| | Treatment | | Mean | (Active/ | Confidence |
| Cycle | Group | N | (×10³/μL) | Placebo) | Interval |
| Cycle 1 | Placebo | 12 | 93.87 | | |
| | RWJ-800088 | 9 | 46.80 | 0.5 | (0.2, 1.2) |
| | 1.5 μg/kg | | | | |
| | RWJ-800088 | 11 | 217.59 | 2.3 | (1.0, 5.4) |
| | 2.25 μg/kg | | | | |
| | RWJ-800088 | 9 | 188.25 | 2.0 | (0.8, 5.1) |
| | 3.0 μg/kg | | | | |
| Cycle 2 | Placebo | 12 | 80.19 | | |
| | RWJ-800088 | 8 | 76.17 | 0.9 | (0.6, 1.6) |
| | 1.5 μg/kg | | | | |
| | RWJ-800088 | 10 | 177.54 | 2.2 | (1.3, 3.6) |
| | 2.25 μg/kg | | | | |
| | RWJ-800088 | 8 | 179.93 | 2.2 | (1.3, 3.9) |
| | 3.0 μg/kg | | | | |
| Across | Placebo | 12 | 62.24 | | |
| | RWJ-800088 | 9 | 34.57 | 0.6 | (0.3, 1.2) |
| | 1.5 μg/kg | | | | |
| | RWJ-800088 | 11 | 165.79 | 2.7 | (1.3, 5.3) |
| | 2.25 μg/kg | | | | |
| | RWJ-800088 | 8 | 134.26 | 2.2 | (1.0, 4.7) |
| | 3.0 μg/kg | | | | |

GMR = geometric mean ratio;
N = number of subjects

TABLE 3
Maximum Platelet Counts in Subjects with
Cancer Receiving Platinum-Based Chemotherapy
(Study NAP1002: All Randomized Subjects Analysis Set)

| | | | Geometric | GMR | 95% |
|---|---|---|---|---|---|
| | Treatment | | Mean | (Active/ | Confidence |
| Cycle | Group | N | (×10³/μL) | Placebo) | Interval |
| Cycle 1 | Placebo | 12 | 299.62 | | |
| | RWJ-800088 | 9 | 329.18 | 1.1 | (0.8, 1.5) |
| | 1.5 μg/kg | | | | |
| | RWJ-800088 | 11 | 564.36 | 1.9 | (1.4, 2.5) |
| | 2.25 μg/kg | | | | |
| | RWJ-800088 | 9 | 642.69 | 2.1 | (1.6, 3.0) |
| | 3.0 μg/kg | | | | |
| Cycle 2 | Placebo | 12 | 314.46 | | |
| | RWJ-800088 | 8 | 294.15 | 0.9 | (0.6, 1.4) |
| | 1.5 μg/kg | | | | |
| | RWJ-800088 | 10 | 401.87 | 1.3 | (0.9, 1.9) |
| | 2.25 μg/kg | | | | |
| | RWJ-800088 | 8 | 434.10 | 1.4 | (0.9, 2.1) |
| | 3.0 μg/kg | | | | |
| Across | Placebo | 12 | 360.22 | | |
| | RWJ-800088 | 9 | 374.76 | 1.0 | (0.8, 1.4) |
| | 1.5 μg/kg | | | | |
| | RWJ-800088 | 11 | 574.27 | 1.6 | (1.2, 2.1) |
| | 2.25 μg/kg | | | | |
| | RWJ-800088 | 8 | 662.94 | 1.8 | (1.3, 2.5) |
| | 3.0 μg/kg | | | | |

GMR = geometric mean ratio;
N = number of subjects
Cross reference: CSR NAP1002 Table 7

Mean platelet counts were lowest on Day 10 following the 2.25 and 3.0 μg/kg doses of RWJ-800088 but continued to decline up to Day 15 after administration of placebo or 1.5 μg/kg of RWJ-800088 in each cycle. Peak mean platelet counts occurred on Day 15 following the 3.0 μg/kg RWJ-800088 dose, whereas the mean peak platelet counts following the lower doses of RWJ-800088 or placebo occurred on Day 21 in both cycles. These data suggest faster recovery of platelet counts at the highest dose of 3.0 μg/kg.

For the 1.5 μg/kg RWJ-800088 dose group and placebo group, the mean platelet counts returned to near baseline levels on Day 21 post dose in both cycles. For the higher dose groups of RWJ-800088 (2.25 and 3.0 μg/kg), the mean platelet counts were higher than baseline levels on Day 21 post dose in both cycles. At the 1.5 μg/kg dose, there was no apparent difference in the platelets as compared with placebo. However, at the 3 μg/kg dose, platelet nadir as well as peak platelet counts were approximately 2-fold higher relative to placebo. The platelet nadir was observed on Day 10 at the 3 μg/kg dose, but for placebo and the 1.5 μg/kg doses, platelets continued to decline up to Day 15. Peak platelets were observed on Day 15 for the 3.0 μg/kg dose, but for placebo and 1.5 μg/kg doses, peak platelets were observed on Day 21. Two subjects at the 3.0 μg/kg dose had a transient platelet increase of more than 3 times the baseline in the first cycle (stopping criteria for further dose escalation). The platelet elevations were attenuated in the second cycle and remained below 3 times the baseline in all subjects. These results at the 3 μg/kg dose indicate reduction in chemotherapy induced decline in platelets and faster recovery relative to placebo and suggest potential for RWJ-800088 in the prevention of chemotherapy induced anemia.

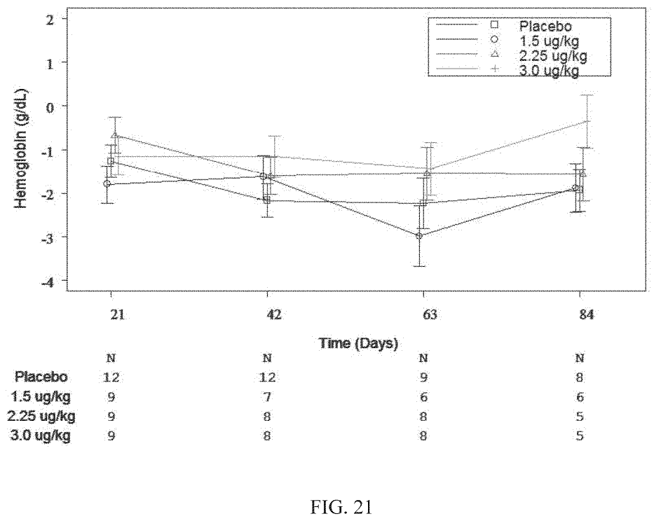

The change in hemoglobin (Hb) concentration values from baseline to the end of Cycle 2 (Day 42) and beyond suggested a dose-related trend for preservation of Hb (Table 5 and FIG. 21). On Day 42, mean Hb concentration had decreased from baseline by 2.17 g/dL in the placebo group, but in the 3.0-μg/kg RWJ-800088 group, the decrease was only 1.16 g/dL, suggesting preservation of Hb by RWJ-800088 treatment. The preservation of Hb in the 3.0-μg/kg RWJ-800088-treated group appears to be sustained beyond the two cycles as indicated by the mean Hb concentrations measured on Day 63 and Day 84 (Table 5).

TABLE 4
Statistical Analysis of Change From Baseline in Hemoglobin at Days 42,
63, and 84 in Subjects With Cancer Receiving Platinum-Based
Chemotherapy
(Study NAP1002: All Randomized Subjects Analysis Set)

| | | | | | Differ- | |
|---|---|---|---|---|---|---|
| | | | | | ence | |
| | | | | | of LS | 95% |
| | | | | Refer- | Mean | Confi- |
| | Treatment | | LS Mean | ence | (SE) | dence |
| Day | Group | N | (SE) (g/dL) | Group | (g/dL) | Interval |
| Day 42 | Placebo | 12 | −2.17 (0.39) | | | |
| | RWJ-800088 | 7 | −1.63 (0.50) | Placebo | 0.54 | (−0.8, |
| | 1.5 μg/kg | | | | (0.63) | 1.8) |
| | RWJ-800088 | 10 | −1.60 (0.42) | Placebo | 0.57 | (−0.6, |
| | 2.25 μg/kg | | | | (0.57) | 1.7) |
| | RWJ-800088 | 8 | −1.16 (0.47) | Placebo | 1.00 | (−0.2, |
| | 3.0 μg/kg | | | | (0.61) | 2.2) |
| Day 63 | Placebo | 9 | −2.23 (0.57) | | | |
| | RWJ-800088 | 6 | −2.98 (0.70) | Placebo | −0.75 | (−2.6, |
| | 1.5 μg/kg | | | | (0.90) | 1.1) |
| | RWJ-800088 | 8 | −1.55 (0.61) | Placebo | 0.68 | (−1.0, |
| | 2.25 μg/kg | | | | (0.83) | 2.4) |
| | RWJ-800088 | 8 | −1.44 (0.61) | Placebo | 0.79 | (−0.9, |
| | 3.0 μg/kg | | | | (0.83) | 2.5) |
| Day 84 | Placebo | 8 | −1.93 (0.51) | | | |
| | RWJ-800088 | 6 | −1.88 (0.59) | Placebo | 0.05 | (−1.6, |
| | 1.5 μg/kg | | | | (0.78) | 1.7) |
| | RWJ-800088 | 7 | −1.90 (0.55) | Placebo | 0.03 | (−1.5, |
| | 2.25 μg/kg | | | | (0.75) | 1.6) |
| | RWJ-800088 | 6 | −0.80 (0.59) | Placebo | 1.13 | (−0.5, |
| | 3.0 μg/kg | | | | (0.78) | 2.7) |

LS = least squares; N = number of subjects; SE = standard error

Translation of the Range of Doses of RWJ-800088 that Provide Increased Survival and Vascular/Organ Protection Based on Platelet Counts and Exposure

Platelets are one of the biomarkers for determining the effective dose of RWJ-800088 for hematopoietic protection and recovery, vascular protection, organ protection, survival or accelerated recovery of vasculature following radiation or chemotherapy exposure. A dose of RWJ-800088 that produced 2-4-fold enhancement of platelets over background in models in mice, rats, dogs, or NHP demonstrated survival, or hematopoietic recovery, or organ or vascular protection, or accelerated vascular recovery. In humans the dose that produced a 3.5-fold enhancement of platelets over background was 3 μg/kg (FIG. 19). Accordingly, the dose of 3 μg/kg is the expected effective for the survival or organ protection, or vascular protection or accelerated vascular recovery in human.

The ratio of the maximum platelet counts to the baseline platelet counts following single escalating doses of RWJ-800088 to mice, rats, dogs, rhesus monkeys and human healthy volunteers is shown in Table 5. The dose required to achieve a 2-3.5-fold elevation in humans is ˜100-fold lower compared to mice, ˜1,000-fold lower compared to rats and >10,000-fold lower compared to canines and NHPs. The maximum platelet elevation was greater than 3-fold for all species except canine is the lease in which the maximum platelet elevation was ˜7.7 fold, suggesting that the canine is the least responsive species to RWJ-800088. The species differences in potency has been described for other TPO mimetics in the literature and is attributed to differences in receptor affinity (Erickson-Miller C L, et al. Discovery and characterization of a selective, nonpeptidyl thrombopoietin receptor agonist. Exp. Hematol. 2005; 33:85-93). Despite the differences in dose, there is clear evidence that comparable maximum platelet response are observed in some species with RWJ-800088. It is discovered that the dose of RWJ-800088 that produces a survival benefit and protects against vascular and organ injury is the dose that produces a 2-4 fold elevation of platelets (Table 6).

TABLE 5
Cross species ratio of the maximum platelet
counts to the baseline platelet counts following
single escalating doses of RWJ-800088.

| | | | | TOX9795/ | |
|---|---|---|---|---|---|
| | 3DP03- | | | B201016- | |
| | Piedmont-01 | TOX6984 | TOX6985 | K013 | NAP 1001 |
| Source: | Mouse Max | Rat Max | Dog Max | NHP Max | Humans Max |
| Dose | Platelet | Platelet | Platelet | Platelet | Platelet |
| (μg/kg) | Ratio | Ratio | Ratio | Ratio | Ratio |
| 0.375 | | | | | 1.4 ± 0.3 |
| 0.75 | | | | | 1.8 ± 0.4 |
| 1.5 | | | | | 1.9 ± 0.7 |
| 2.25 | | | | | 2.1 ± 0.8** |
| 3 | | | | | 3.1 ± 1.1** |
| 10 | 0.9 ± 0.6 | | | | |
| 30 | 1.8 ± 0.4 | | | | |
| 100 | 2.5 ± 1.1** | 1.0 ± 0.2 | | | |
| 300 | 3.9 ± 0.9** | 1.7 ± 0.3 | 1.0 ± 0.3 | | |
| 500 | | | | 0.9 ± 0.1 | |
| 1000 | 4.3 ± 0.5** | | | | |
| 2000 | | | | 1.1 ± 0.1 | |
| 3000 | 3.4 ± 1** | 3.2 ± 0.6** | 1.4 ± 0.5 | | |
| 10000 | | | 1.7 ± 0.5* | 2.9 ± 0.0 | |
| 30000 | | | | 3.25*\hat{ } | |
| 40000 | | | | 3.8 ± 0.1 | |

The doses that produced modest to large beneficial effects on survival are bolded and
** = large pharmacodynamics (PD) effect and
* = modest effect
\hat{ }CiToxLAB North America Study No. 2016-2693

TABLE 6
Summary of the doses for rats, mice, non-human primates and humans that
produce a therapeutic benefit across pharmacology models relative to the
doses that produce a 2.5-4x elevation of platelets. These results demonstrate
a consistent trend for achieving vascular protection and survival benefit across
species and that a dose of 3 μg/kg would be a preferred dose
for RWJ-800088 in humans.

| | | Doses and Relative | Dose for 2.0- | |
|---|---|---|---|---|
| | | Pharmacodynamic | 4x Platelet | |
| | | Effect (Bold | Elevation in | |
| | | Indicates Effect | Healthy | |
| Species | Study | Observed) | Subjects | Effects |
| Humans | Phase 1b Study | 1 < 2.25 = 3 μg/kg | ≥2.25 μg/kg | Protection against |
| | in Cancer | | | thrombocytopenia and |
| | Patients | | | anemia |
| | receiving | | | |
| | carboplatin | | | |
| | chemotherapy | | | |
| Mice | Carboplatin | 0.03 << 0.1 = 0.3 | ≥0.1 mg/kg | Protection against |
| | chemotherapy | mg/kg | | thrombocytopenia, |
| | | | | anemia and vascular |
| | | | | hemorrhage in the brain |
| Mice | Whole body | 0.1 < 0.3, 1, > 2, 3 | ≥0.1 mg/kg | Enhanced survival, |
| | irradiation | mg/kg | | mitigation against |
| | | | | thrombocytopenia, |
| | | | | anemia, reduced adhesion |
| | | | | protein expression on |
| | | | | endothelial cells |
| Mice | Ear vein | 0.3 mg/kg | ≥0.1 mg/kg | Reduced white cell |
| | | | | adhesion to endothelium |
| | | | | and vascular permeability |
| Rats | Whole body | 0.3 < 3 mg/kg | ≥3 mg/kg | Enhanced survival, |
| | irradiation | | | mitigation against |
| | | | | thrombocytopenia, |
| | | | | anemia |
| Rats | Prostate | 3 mg/kg | ≥3 mg/kg | Protection of vascular size |
| | Irradiation | | | and function |
| Rhesus | Whole body | 30 mg/kg | ≥30 mg/kg | Enhanced survival, |
| | irradiation | | | mitigation of |
| | | | | thrombocytopenia, |
| | | | | anemia |

### Example 4: Dose Reduction Factor (DRF) Study of TPOm on Survival Following Exposure to Different Doses of Irradiation in Mice

Methods: CD21F1 male mice were used in a DRF (dose reduction factor) study to determine LD50/30 for animals administered with RWJ-800088 or it's vehicle (saline). This included irradiating cohorts of 24 animals at various total body irradiation (TBI) doses (Saline: 7.5, 8.0, 8.5, 9.0, 9.5, and 10 Gy; RWJ-800088: 10.5, 11.0, 11.5, 11.75, 12, and 12.5 Gy). Following the 30 day survival study, surviving animals were monitored for up to 1 year with planned collection points at 6 months and 1 year.

RWJ-800088 administration timing and doses: RWJ-800088 was administered RWJ-800088 was administered at a dose of 1 mg/kg 24 h prior to irradiation.

Results

Survival following TBI: Survival Data are listed in Table 8 below.

TABLE 8

| | | | # | # | |
|---|---|---|---|---|---|
| Group | Radiation | Drug/ | Surviving | Surviving | # Surviving |
| # | Dose | Saline | (30 days) | (6 months) | (12 months) |
| 1 | 0 Gy | No | 15/15 | 6/6 | 3/3 |
| | | | (100%) | (100%) | (100%) |
| 5 | 9.5 Gy | Saline | 12/24 | 08/10 | 3/4 up to |
| | | | (50%) | (80%) | 8 Months. |
| | | | | | Then euthanized |
| | | | | | due to MPV |
| | | | | | infection |
| 7 | 10.5 Gy | TPOm | 24/24 | 10/10 | 5/5 |
| | | | (100%) | (100%) | (100%) |
| 9 | 11.5 Gy | TPOm | 22/24 | 10/10 | 5/5 |
| | | | (92%) | (100%) | (100%) |
| 12 | 12.5 Gy | TPOm | 11/24 | 7/10 | 0/3 |
| | | | (46%) | (70%) | (0%) |
| | | | | Two animals | |
| | | | | were added | |
| | | | | from 12 Gy | |

Animals administered RWJ-800088 prior to irradiation at up to 11.5 Gy survived the duration of the study (aside from planned sacrifices) whereas mortality was observed in the animals administered the vehicle prior to irradiation at 9.5 Gy.

Analysis of the survival data yielded a DRF value of 1.42 (95% CI 1.16-2.54) and demonstrated a significant increase in survival. The analysis of the survival data also yielded the LD50 value for saline is 8.93 Gy and the LD50 value for RWJ-800088 is 12.64 Gy.

The above data demonstrated that RWJ-800088 administration can provide protective survival benefits up to 12 months.

### Example 5: Modulation Effects of TPOm on Blood Cells and Bone Marrow Following Exposure to Irradiation in Mice

Methods: During the above Experiment 4, four studies were performed as follows:

Study A: A subset of the animals were sacrificed at 1, 6, and 12 months. Blood was collected and blood cells counted, and femurs were collected, bone marrow isolated and cultured to analyze for colony forming units;

Study B: A subset of the animals were sacrificed at 1 and 6 months. Sterna were collected from these animals, fixed, sectioned, stained with H&E (Hematoxylin and eosin), and megakaryocytes counted;

Study C: A subset of the animals were sacrificed at 1 and 6 months. Kidneys were collected from these animals, fixed, sectioned, stained with β-Catenin or E-cadherin: and

Study D: A subset of the animals were sacrificed at 1 and 6 months. Kidneys were collected from these animals, fixed, sectioned, stained with β-galactosidase, a marker of senescence.

Results

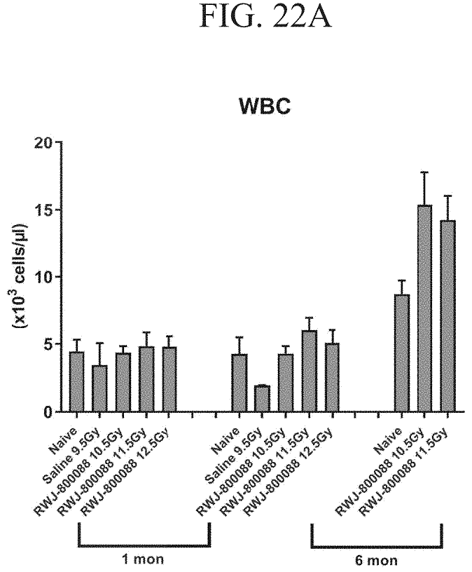

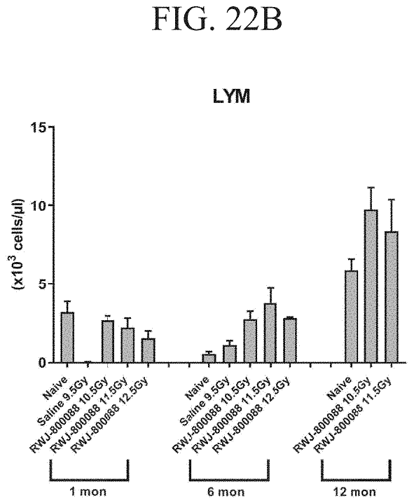

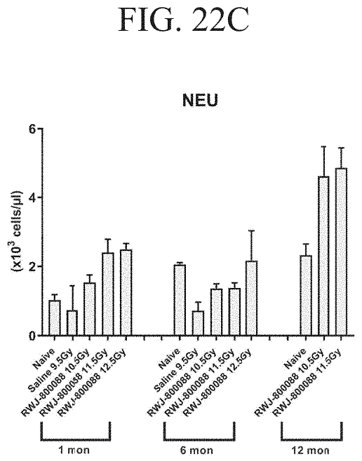

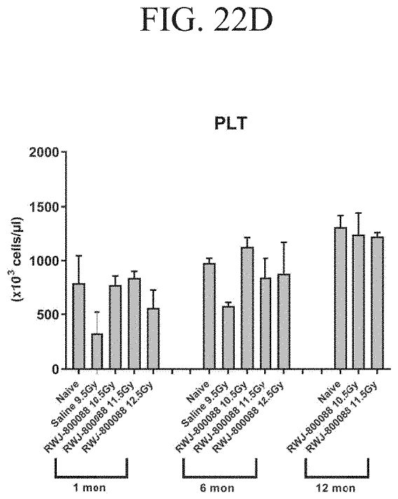

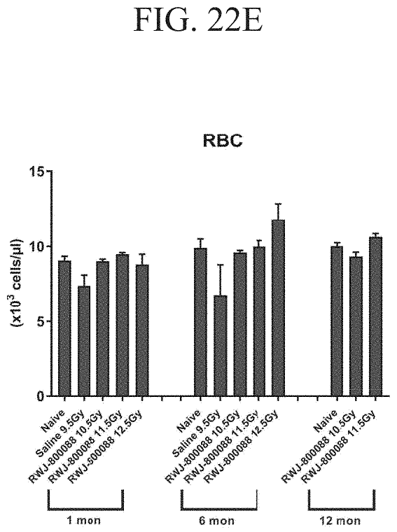

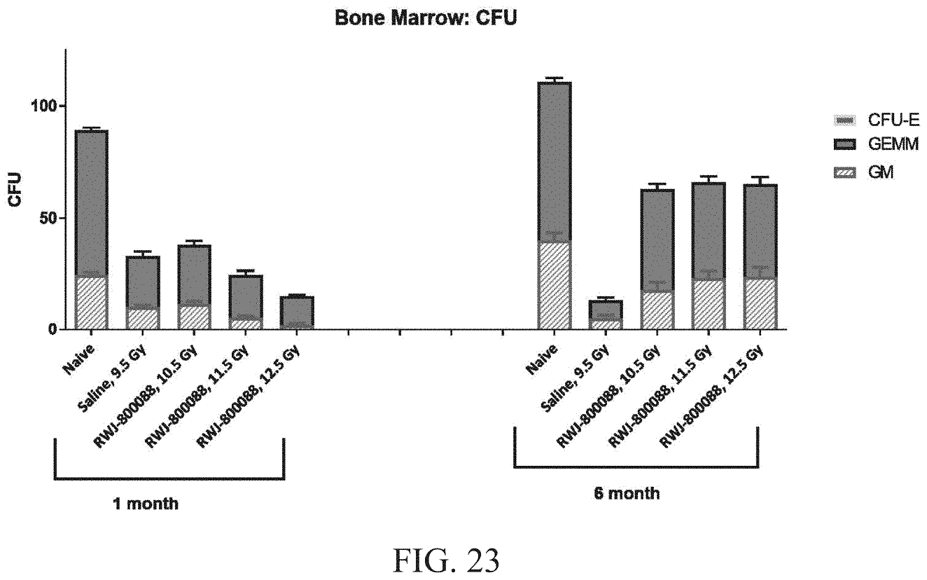

Study A: As shown in FIGS. 22A-E and FIGS. 23A-B, several blood cell types were increased in number for animals administered with RWJ-800088 compared to those administered vehicle at 6 months and 12 months post TBI including white blood cells (FIG. 22A), lymphocytes (FIG. 22B), neutrophils (FIG. 22C), platelets (FIG. 22D), and red blood cells (FIG. 22E). Animals administered RWJ-800088 also had more colony forming units in isolated bone marrow (FIG. 23). Those data and figures demonstrated that RWJ-800088 administration can increase cell counts and ability of bone marrow to form colonies in long term survivors (up to 6 months).

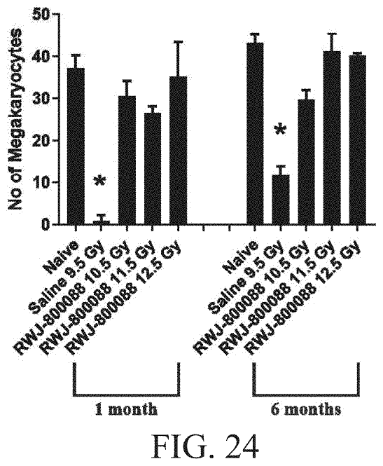

Study B: As shown in FIG. 24, significantly higher numbers of megakaryocytes were observed in animals administered RWJ-800088 compared to its vehicle (saline) at both 1 and 6 months post irradiation, which indicates that RWJ-800088 administration can increase megakaryocyte abundance in long term survivors (up to 6 months).

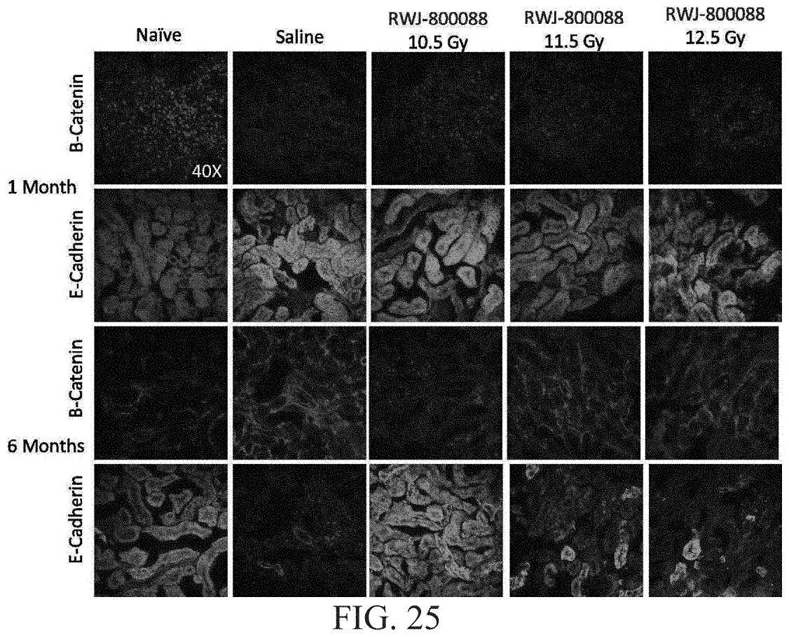

Study C: As shown in FIG. 25, β-Catenin (red on first and third rows) expression was higher in animals administered the vehicle (saline) than in animals administered RWJ-800088. E-cadherin expression (green on second and fourth rows) was higher in animals administered RWJ-800088 compared to those administered vehicle. Those data demonstrated that RWJ-800088 administration can increase E-cadherin expression and decreases β-Catenin expression in long term survivors (up to 6 months).

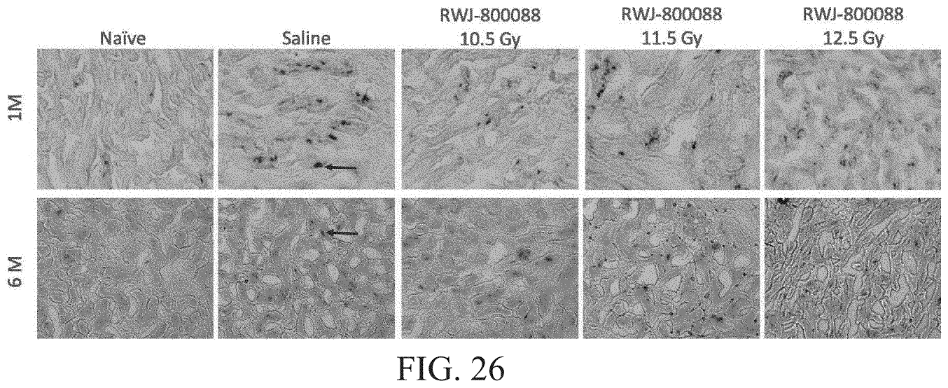

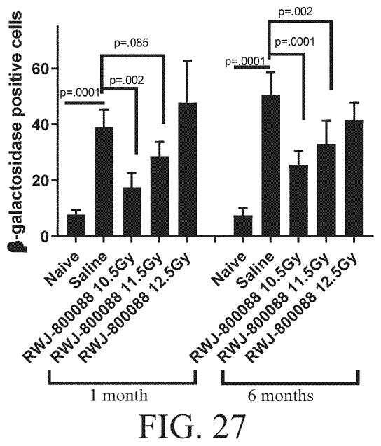

Study D: As shown in FIG. 26 and FIG. 27, more cells stained positive for β-galactosidase (dark spots) in animals administered vehicle compared to those administered RWJ-800088, which indicates that RWJ-800088 administration can protects from cellular senescence in long term survivors (up to 6 months).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of mitigating vascular injury, promoting organ and/or hematopoietic recovery, enhancing survival, and/or protecting against organ and hematopoietic injury in a human subject that may be or has been exposed to radiation or radiomimetic agents as a consequence of an attack or accident or that may be treated with at least one of a radiation therapy and a radiomimetic chemotherapy, the method comprising subcutaneously administering to the human subject an effective amount of a thrombopoietin (TPO) mimetic comprising the amino acid sequence of SEQ ID NO:1, wherein the effective amount comprises 0.1 microgram (μg) to 6 μg, preferably 2.25 μg to 4 μg, of the TPO mimetic per kilograms (kg) body weight of the subject, or a fixed or tiered dose equivalent based upon a typical body weight of the subject population.

2. A method of treating a human subject in need of eradication of malignant cells and/or suppression of immune system, comprising:
(a) treating the human subject with at least one of a radiation therapy and a radiomimetic chemotherapy, and
(b) subcutaneously administering to the human subject an effective amount of a thrombopoietin (TPO) mimetic comprising the amino acid sequence of SEQ ID NO:1,
wherein the effective amount comprises 0.1 microgram (μg) to 6 μg, preferably 2.25 μg to 4 μg, of the TPO mimetic per kilograms (kg) body weight, or a fixed or tiered dose equivalent based upon a typical body weight of the subject population.

3. The method of claim 1 or 2, wherein the TPO mimetic is administered to the subject within about 32 hours prior to and about 24 hours after the subject being treated with the at least one of a radiation therapy and a radiomimetic chemotherapy.

4. A method of mitigating vascular injury, promoting organ and/or hematopoietic recovery, enhancing survival, and/or protecting against organ and hematopoietic injury in a subject treated with at least one of a radiation therapy and a radiomimetic chemotherapy, the method comprising administering to the subject an effective amount of a thrombopoietin (TPO) mimetic comprising the amino acid sequence of SEQ ID NO:1, wherein the TPO mimetic is administered to the subject within about 32 hours prior to the subject being treated with the at least one of the radiation therapy and the radiomimetic chemotherapy.

5. A method of treating a subject in need of eradication of malignant cells and/or suppression of immune system, comprising:
(a) treating the human subject with at least one of a radiation therapy and a radiomimetic chemotherapy, and
(b) administering to the subject an effective amount of a thrombopoietin (TPO) mimetic comprising the amino acid sequence of SEQ ID NO:1,
wherein the TPO mimetic is administered to the subject within about 32 hours prior to the subject being treated with the at least one of the radiation therapy and the radiomimetic chemotherapy

6. The method of claim 4 or 5, wherein the effective amount of TPO mimetic is administered to the subject subcutaneously, and
when the subject is a human being, the effective amount of the TPO mimetic is about 0.1 microgram (μg) to about 6 μg, preferably 2.25 μg to 4 μg, of the TPO mimetic per kilograms (kg) body weight of the subject, or a fixed or tiered dose equivalent based upon a typical body weight of the subject population;
when the subject is a mouse, the effective amount of the TPO mimetic is about 100 μg to about 5000 μg/kg body weight of the subject;
when the subject is a rat, the effective amount of the TPO mimetic is about 1000 μg to about 50,000 μg/kg body weight of the subject; or
when the is a dog or a monkey, the effective amount of the TPO mimetic is about 10,000 μg to about 500,000 μg/kg body weight of the subject.

7. The method of any one of claims 1 to 6, wherein the TPO mimetic is RWJ-800088 having the following structure of formula (I), or a pharmaceutically acceptable salt or ester thereof: 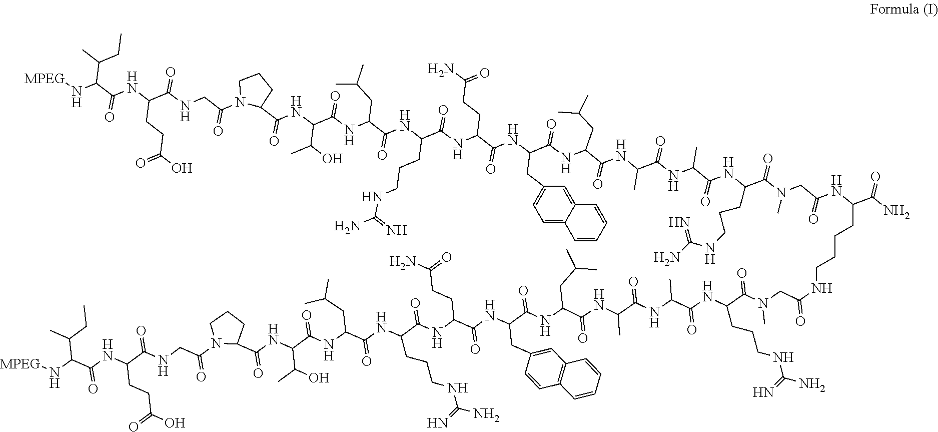
wherein MPEG represents methoxypolyethyleneglycol20000.

8. The method of any one of claims 1 to 6, wherein the TPO mimetic is romiplostim comprising the amino acid sequence of SEQ ID NO:4.

9. The method of any one of claims 1 to 8, wherein the subject is treated for a cancer selected from the group consisting of prostate cancer, head and neck cancer, hepatocellular carcinoma, colon cancer, lung cancer, melanoma, pancreatic, and breast cancer, and the subject is treated with a targeted radiation therapy.

10. The method of any one of claims 1 to 8, wherein the subject is treated for a cancer selected from the group consisting of a leukemia, a solid tumor, Morbus Hodgkin's disease and Non-Hodgkin's lymphomas, and the subject is treated with total body irradiation prior to a transplantation of at least one of haematopoietic stem cells, bone marrow stem cells and peripheral blood progenitor stem cells.

11. The method of any one of claims 1 to 10, wherein the subject is treated with a radiomimetic chemotherapy selected from the group consisting of ozone, peroxide, an alkylating agent, a platinum-based agent, a cytotoxic antibiotic, and a vesicant chemotherapy, preferably, the radiomimetic chemotherapy is cyclophosphamide, busulfan, fludarabine, melphalan, thiotepa, cytarabine and clofarabine, carmustine, etoposide, cytarabine and melphalan, Rituximab, ifosfamide, etoposide, or a platinum-based agent selected from the group consisting of cisplatin, carboplatin, oxaliplatin, and nedaplatin.

12. The method of any one of claims 1 to 11, wherein the subject is administered a single dose of the effective amount of the TPO mimetic.

13. The method of any one of claims 1 to 11, wherein the subject is administered more than one dose of the effective amount of the TPO mimetic.

14. A method of treating a cancer in a human subject in need of, comprising:
(a) treating the human subject with at least one of a radiation therapy and a radiomimetic chemotherapy, and
(b) subcutaneously administering to the human subject an effective amount of a thrombopoietin (TPO) mimetic comprising RWJ-800088 or romiplostim,
wherein the effective amount comprises 0.5 microgram (μg) to 5 μg, preferably 2.25 μg to 4 μg, of the TPO mimetic per kilograms (kg) body weight of the subject, or a fixed or tiered dose equivalent based upon a typical body weight of the subject population, and the TPO mimetic is administered to the subject within about 32 hours prior to the subject being treated with the at least one of the radiation therapy and the radiomimetic chemotherapy.

15. The method of claim 14, wherein the subject is treated for a cancer selected from the group consisting of prostate cancer, head and neck cancer, hepatocellular carcinoma, colon cancer, lung cancer, melanoma, pancreatic, and breast cancer, and the subject is treated with a targeted radiation therapy.

16. The method of claim 14, wherein the subject is treated for a cancer selected from the group consisting of a leukemia, multiple myeloma, a solid tumor, Morbus Hodgkin's disease and Non-Hodgkin's lymphomas, and the subject is treated with total body irradiation prior to a transplantation of at least one of hematopoietic stem cells, bone marrow stem cells and peripheral blood progenitor stem cells.

17. The method of any one of claims 14 to 16, wherein the subject is treated with a radiomimetic chemotherapy selected from the group consisting of ozone, peroxide, an alkylating agent, a platinum-based agent, a cytotoxic antibiotic, and a vesicant chemotherapy, preferably, the radiomimetic chemotherapy is cyclophosphamide, busulfan, fludarabine, melphalan, thiotepa, cytarabine and clofarabine, carmustine, etoposide, cytarabine and melphalan, Rituximab, ifosfamide, and etoposide or a platinum-based agent selected from the group consisting of cisplatin, carboplatin, oxaliplatin, and nedaplatin.

18. The method of any one of claims 14 to 17, wherein the subject is administered a single dose of the effective amount of the TPO mimetic.

19. The method of any one of claims 14 to 17, wherein the subject is administered more than one dose of the effective amount of the TPO mimetic.
